(19) United States
(12) Patent Application Publication
(10) Pub. No.: US 2025/0186363 A1
(43) Pub. Date: Jun. 12, 2025

(54) Pharmaceutical Compositions and Methods for Treating Parkinson's Disease

(71) Applicant: Pike Therapeutics Inc., Vancouver (CA)

(72) Inventors: Fotios M. PLAKOGIANNIS, Whitestone, NY (US); Nisarg MODI, Jersey City, NJ (US)

(21) Appl. No.: 19/053,616

(22) Filed: Feb. 14, 2025

Related U.S. Application Data
(63) Continuation of application No. 17/401,374, filed on Aug. 13, 2021.
(60) Provisional application No. 63/229,021, filed on Aug. 3, 2021.
(60) Provisional application No. 63/066,386, filed on Aug. 17, 2020.

(51) Int. Cl. A61K 9/70; A61K 9/00; A61K 31/05; A61K 31/197; A61K 31/352; A61K 31/4045; A61K 31/428; A61K 31/473; A61K 31/4745; A61K 31/4985; A61P 25/16

(52) U.S. Cl. CPC A61K 9/7069; A61K 9/0021; A61K 9/7023; A61K 31/05; A61K 31/197; A61K 31/352; A61K 31/4045; A61K 31/428; A61K 31/473; A61K 31/4745; A61K 31/4985; A61P 25/16

(57) ABSTRACT

Pharmaceutical compositions and methods for treating and/or preventing and/or control of Parkinson's Disease and/or related symptoms in a patient.

This application is a continuation of U.S. Ser. No. 17/401,374 filed Aug. 13, 2021, which claims benefit of U.S. Ser. No. 63/066,386, filed Aug. 17, 2020, and U.S. Ser. No. 63/229,021, filed Aug. 3, 2021, the entireties of which are incorporated herein by reference.

### SPECIFICATION

### Background

Parkinson's disease (PD) is a degenerative disorder of the central nervous system mainly affecting the motor system and belongs to a group of conditions called motor system disorders, which are the result of the loss of dopamine-producing cells in the substantia nigra. The causes of this cell death are largely unknown. Early in the course of the disease, the most obvious symptoms are movement-related; these include shaking, rigidity, slowness of movement and difficulty with walking and gait. Later, thinking and behavioral problems may arise, with dementia commonly occurring in the advanced stages of the disease. Depression is the most common psychiatric symptom. Other symptoms include sensory, sleep and emotional problems. Parkinson's disease is more common in older people, with most cases occurring after the age of 50; when it is seen in young adults, it is called Young Onset PD (YOPD).

The four primary symptoms of PD are tremor, or trembling in hands, arms, legs, jaw, and face; rigidity, or stiffness of the limbs and trunk; bradykinesia, or slowness of movement; and postural instability, or impaired balance and coordination. As these symptoms become more pronounced, patients may have difficulty walking, talking, or completing other simple tasks. Other symptoms may include depression and other emotional changes; difficulty in swallowing, chewing, and speaking; urinary problems or constipation; skin problems; and sleep disruptions. There are currently no blood or laboratory tests that have been proven to help in diagnosing sporadic PD, and diagnosis is based on medical history and a neurological examination.

The main motor symptoms are collectively called parkinsonism, or a “parkinsonian syndrome.” The disease can be either primary or secondary. Primary Parkinson's disease is referred to as idiopathic (having no known cause), although some atypical cases have a genetic origin, while secondary parkinsonism is due to known causes like toxins. Many risks and protective factors have been investigated: the clearest evidence is for an increased risk of PD in people exposed to certain pesticides and a reduced risk in tobacco smokers. The pathology of the disease is characterized by the accumulation of a protein into Lewy bodies in neurons, and insufficient formation and activity of dopamine in certain parts of the midbrain. Where the Lewy bodies are located is often related to the expression and degree of the symptoms of an individual. Diagnosis of typical cases is mainly based on symptoms, with tests such as neuroimaging being used for confirmation.

Currently, there is no cure for PD, but a variety of medications provide relief from the symptoms. Treatments, typically the medications L-DOPA and dopamine agonists, improve the early symptoms of the disease. As the disease progresses and dopaminergic neurons continue to be lost, these drugs eventually become ineffective at treating the symptoms and at the same time produce a complication marked by involuntary writhing movements. Diet and some forms of rehabilitation have shown some effectiveness at improving symptoms. Surgery and deep brain stimulation have been used to reduce motor symptoms as a last resort in severe cases where drugs are ineffective. Research directions include investigations into new animal models of the disease and of the potential usefulness of gene therapy, stem cell transplants and neuroprotective agents. Medications to treat non-movement-related symptoms of PD, such as sleep disturbances and emotional problems, also exist.

Levodopa (L-DOPA) has been the most widely used treatment since 1967. L-DOPA is converted into dopamine in the dopaminergic neurons by the enzyme dopa decarboxylase. Since motor symptoms are produced by a lack of dopamine in the substantia nigra, the administration of L-DOPA temporarily diminishes the motor symptoms. Usually, affected individuals are given levodopa combined with carbidopa, which delays the conversion of levodopa into dopamine until it reaches the brain. Nerve cells can use levodopa to make dopamine and replenish the brain's dwindling supply. Although levodopa helps at least three-quarters of parkinsonian cases, not all symptoms respond equally to the drug. Bradykinesia and rigidity respond best, while tremor may be only marginally reduced. Problems with balance and other symptoms may not be alleviated at all. Anticholinergics may help control tremor and rigidity. Other drugs, such as bromocriptine, pramipexole, and ropinirole, mimic the role of dopamine in the brain, causing the neurons to react as they would to dopamine. An antiviral drug, amantadine, also appears to reduce symptoms.

An estimated 53 million people have PD resulting in about 103,000 deaths globally. Parkinson's disease typically occurs in people over the age of 60, with males being affected more often than females. The average life expectancy following diagnosis is between 7 and 14 years. Parkinson's disease is typically idiopathic (having no specific known cause); however, a proportion of cases can be attributed to known genetic factors. For example, mutations in specific genes have been shown to cause PD (e.g., alpha-synuclein (SNCA), parkin (PRKN), leucine-rich repeat kinase 2 (LRRK2), PTEN-induced putative kinase 1 (PINK1), DJ-1 and ATP13A2). Mutations in LRRK2 are the most common known cause of familial and sporadic PD, accounting for approximately 5% of individuals with a family history of the disease and 3% of sporadic cases. The LRRK2 G2019S gain of function gene mutation is one of the most prevalent mutations contributing to PD pathogenesis. While treatments for PD are available, more effective therapies are needed.

Modern treatments try to manage the early motor symptoms of the disease, mainly through the use of levodopa and dopamine agonists. As the disease progresses and dopamine neurons continue to be lost, a point eventually arrives at which these drugs become ineffective at treating the symptoms and at the same time produce a complication called dyskinesia, marked by involuntary writhing movements. Therefore, there is a need in the art to be able to deliver large oral equivalent dose of, for example, CBD to obtain a therapeutic effect, and eliminate or reduce discontinuance, adverse events, and side effects, such as diarrhea, somnolence, abdominal pain, weight gain, headaches, etc., as adverse events like liver enzyme elevations, associated with large oral doses to treat motor symptoms in subjects with Parkinson's Disease, including symptoms of Parkinson's Disease as well as symptoms indirectly associated with Parkinson's Disease, such as those arising as side effects of treatment.

The present disclosure provides compositions and methods for the treatment and/or prevention and/or control of Parkinson's Disease in a patient comprising administration of, for example, CBD and/or THC, as set forth herein.

Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannbinol (CBN), cannabichromene (CBC), cannabigerol (CBG), tetrahydrocannbivarin (THCV), delta 9-tetrahydrocannabinol (delta9THC) and many more. It has been showed that cannabis and its derivatives can be used for the treatment of pain, type-2 related metabolic disorder, decrease intraocular pressure, Dravet syndrome, Lennox-Gastaut Syndrome (LGS), epilepsy, nausea, pain and wasting associated with AIDS, arthritis and rheumatism, migraines, muscle spasticity associated with multiple sclerosis and paralysis, alcohol and narcotics withdrawal, stress and depression, asthma, fibromyalgia, inflammatory pain, and pain and/or inflammation associated with chemotherapy, act as an antimicrobial. FDA approved Marinol and Syndros contains delta 9-THC, which currently used in treatment of nausea, vomiting, and anorexia associated with chemotherapy treatments. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation. (Costa, B. The non-psychoactive cannabis constituent cannabidiol is an orally effective therapeutic agent in rat chronic inflammatory and neuropathic pain. European Journal of Pharmacology. Volume 556, Issues 1-3, 5 Feb. 2007, Pages 75-83).

Currently, 107 different cannabinoids have been identified from cannabis Sativa. These compounds are similar to the endogenous cannabinoid group that consists of long chain polyunsaturated fatty acids⁴. There are two types of cannabinoid receptors: I) CB1 and II) CB2. The endocannabinoid system is a one of the important endogenous lipids signaling pathway, which consists of cannabinoid receptors, the endogenous ligands of cannabinoid receptors (endocannabinoids) and the enzymes that regulate the biosynthesis and inactivation of endocannabinoids. The lipid signaling system is involved in many important physiological functions in the central and peripheral nervous system and in the endocrine and immune system. These receptors are from family of Guanosine Binding Protein-Coupled Receptors, are widely expressed and distinguished by their specific function, localization and signaling mechanisms^{5,3}. Cannabinoid receptor 1 (CB1), inhibits adenylate cyclase and reduce cAMP levels and protein kinase A (PKA) activity, resulting in the activation of the A-type potassium channels and decrease cellular potassium levels. This receptor mainly found in adipose tissue, the GI tract, the spinal cord, the adrenal and thyroid glands, liver, reproductive organ and immune cells.

Cannabidiol (CBD), a major non-psychoactive phytocannabinoid has little affinity for CB1 and CB2, and act as a partial antagonist CB1 and as a weak inverse CB2 agonist.

Many orally delivered drugs irritate the gastrointestinal mucosa and a large number, including CBD, undergo extensive ‘first-pass’ inactivation by the liver. The compositions and methods of the disclosure are directed to drug delivery directly to the systemic circulation via application to the skin, and thus the compositions and methods of the disclosure overcome these problems because the active agent avoids being metabolized by the liver after absorption, and also gastrointestinal irritation is avoided.

These side effects related to oral drugs can be avoided using transdermal route. Furthermore, the peak and valley in the plasma concentration due to oral administration can be avoided by delivering the drug molecule constantly at predetermined input rate using transdermal dosage forms.

In addition, the present disclosure provides compositions and methods have the unique benefit of allowing a patient or health care provider to immediately stop drug administration by simply removing the patch. For example, a patient may start with a dose that has the onset of unwanted negative side effects. The patient or heath care provider has the ability to control or stop administration (remove patch) before it gets worse, and also dramatically shorten the time of the unwanted experience.

All references cited herein are incorporated herein by reference in their entireties.

### BRIEF SUMMARY

The disclosure provides a method of treating, preventing, and/or slowing the onset or progression of Parkinson's disease (PD) and/or a related symptom in a patient comprising: selecting a patient in need of treating, preventing, and/or slowing the onset or progression of PD and/or a related symptom; topically applying a pharmaceutical composition comprising at least one active agent selected from the group consisting of: about 0.1% to about 50% of an active agent selected from the group consisting of cannabidiol (CBD), free base forms thereof, salts thereof, isomers thereof, amorphous forms thereof, derivatives thereof, and combinations thereof, about 0.1% to about 50% of an active agent selected from the group consisting of tetrahydrocannabinol (THC), free base forms thereof, salts thereof, isomers thereof, amorphous forms thereof, derivatives thereof, and combinations thereof, and combinations thereof, further wherein the pharmaceutical composition comprises: about 1% to about 99% of at least one solvent; about 1% to about 99% of at least surfactant; optionally, about 1% to about 99% of at least one permeation enhancer; and/or optionally, about 30% to about 99% of an adhesive and/or polymer. The disclosure provides a method of treating, preventing, and/or slowing the onset or progression of Parkinson's disease (PD) and/or a related symptom in a patient wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, and about 5 μg/mL. The disclosure provides a method wherein the active agent is present at a concentration selected from the group consisting of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, and about 50% of the formulation. The disclosure provides a method wherein the active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation. The disclosure provides a method of treating, preventing, and/or slowing the onset or progression of Parkinson's disease (PD) and/or a related symptom in a patient wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a method of treating, preventing, and/or slowing the onset or progression of Parkinson's disease (PD) and/or a related symptom in a patient wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a method of treating, preventing, and/or slowing the onset or progression of Parkinson's disease (PD) and/or a related symptom in a patient wherein the pharmaceutical composition comprises one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery. The disclosure provides a method of treating, preventing, and/or slowing the onset or progression of Parkinson's disease (PD) and/or a related symptom in a patient wherein the pharmaceutical composition is formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, or transdermal drug-in-adhesive matrix formulation. The disclosure provides a method of treating, preventing, and/or slowing the onset or progression of Parkinson's disease (PD) and/or a related symptom in a patient wherein the pharmaceutical composition is formulated as a topical liquid formulation, topical semisolid formulation, topical gel formulation, topical polymer matrix formulation, topical adhesive matrix formulation, topical film forming gel formulation, or topical film forming spray formulation. The disclosure provides a method of treating, preventing, and/or slowing the onset or progression of Parkinson's disease (PD) and/or a related symptom in a patient wherein the pharmaceutical composition further comprises carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof. The disclosure provides a method of treating, preventing, and/or slowing the onset or progression of Parkinson's disease (PD) and/or a related symptom in a patient wherein the pharmaceutical composition further comprises carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v. The disclosure provides a method of treating, preventing, and/or slowing the onset or progression of Parkinson's disease (PD) and/or a related symptom in a patient wherein the pharmaceutical composition is formulated as a transdermal patch. The disclosure provides a method of treating, preventing, and/or slowing the onset or progression of Parkinson's disease (PD) and/or a related symptom in a patient wherein the pharmaceutical composition is formulated as a multilayer transdermal matrix system, a metered dose transdermal gel, metered dose transdermal spray, a film forming gel, a film forming spray, or a meter-dose aerosol. The disclosure provides a method of treating, preventing, and/or slowing the onset or progression of Parkinson's disease (PD) and/or a related symptom in a patient wherein the pharmaceutical composition is formulated as a topical patch. The disclosure provides a method of treating, preventing, and/or slowing the onset or progression of Parkinson's disease (PD) and/or a related symptom in a patient wherein the pharmaceutical composition is formulated as metered dose gel, metered dose spray, gel, cream, solution, emulsion, liquid compositions, semisolid compositions, a matrix of adhesive in combination with polymers, or film forming formulations. The disclosure provides a method of treating, preventing, and/or slowing the onset or progression of Parkinson's disease (PD) and/or a related symptom in a patient wherein the pharmaceutical composition is formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof. The disclosure provides a method of treating, preventing, and/or slowing the onset or progression of Parkinson's disease (PD) and/or a related symptom in a patient wherein the pharmaceutical composition is formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof. The disclosure provides a method of treating, preventing, and/or slowing the onset or progression of Parkinson's disease (PD) and/or a related symptom in a patient wherein the pharmaceutical composition is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a method of treating, preventing, and/or slowing the onset or progression of Parkinson's disease (PD) and/or a related symptom in a patient wherein the pharmaceutical composition is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a method of treating, preventing, and/or slowing the onset or progression of Parkinson's disease (PD) and/or a related symptom in a patient wherein the pharmaceutical composition is formulated as microneedles. The disclosure provides a method of treating, preventing, and/or slowing the onset or progression of Parkinson's disease (PD) and/or a related symptom in a patient wherein the said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route. The disclosure provides a method of treating, preventing, and/or slowing the onset or progression of Parkinson's disease (PD) and/or a related symptom in a patient wherein the active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition. The disclosure provides a method of treating, preventing, and/or slowing the onset or progression of Parkinson's disease (PD) and/or a related symptom in a patient wherein the pharmaceutical composition is co-administered with at least one additional an active agent selected from the group consisting of: a dopamine precursor, a dopamine receptor agonist, and combinations thereof. The disclosure provides a method of treating, preventing, and/or slowing the onset or progression of Parkinson's disease (PD) and/or a related symptom in a patient wherein the pharmaceutical composition is co-administered with at least one additional an active agent selected from the group consisting of: levodopa, bromocriptine, pergolide, pramipexole, cabergoline, ropinorole, apomorphine, and any combination thereof.

The disclosure provides a method of treating, preventing, and/or slowing the onset or progression of Parkinson's disease (PD) and/or a related symptom in a patient wherein the symptom of PD to be treated, prevented, and/or slowed is selected from the group consisting of: (a) at least one non-motor aspect of experiences of daily living as defined by Part I of the Unified Parkinson's Disease Rating Scale selected from the group consisting of cognitive impairment, hallucinations and psychosis, depressed mood, anxious mood, levodopa induced nausea, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation;
(f) depression; (g) cognitive impairment; (h) short or long term memory impairment; (i) concentration impairment; (j) coordination impairment; (k) mobility impairment; (l) speech impairment; (m) mental confusion; (n) sleep problem, sleep disorder, or sleep disturbance; (o) circadian rhythm dysfunction; (p) hallucinations; (q) fatigue; (r) REM disturbed sleep; (s) REM behavior disorder; (t) erectile dysfunction; (u) postural hypotension; (v) correction of blood pressure or orthostatic hypotension; (w) nocturnal hypertension; (x) regulation of temperature; (y) improvement in breathing or apnea; (z) correction of cardiac conduction defect; (aa) amelioration of pain; (bb) urinary incontinence, or restoration of bladder sensation and urination; (cc) mood swings; (dd) apathy; (ee) control of nocturia; (ff) neurodegeneration; (gg) anxiety, including anxiety related to public speaking; (hh) improving speaking ability, including public speaking ability; and/or (ii) Parkinson's Disease Dementia.

The disclosure provides a method of treating, preventing, and/or slowing the onset or progression of Parkinson's disease (PD) and/or a related symptom in a patient wherein the PD symptom to be treated, prevented, and/or slowed is a sleep problem, sleep disorder, sleep disturbance, circadian rhythm dysfunction, REM disturbed sleep, or REM behavior disorder, and wherein: (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof, and/or (b) the REM-behavior disorder comprises vivid dreams, nightmares, and acting out the dreams by speaking or screaming, or fidgeting or thrashing of arms or legs during sleep; and/or (c) treating the sleep problem, sleep disorder, sleep disturbance prevents or delays the onset and/or progression of the Parkinson's disease; and/or (d) the method results in a positive change in the sleeping pattern of the subject over a defined period of time; and/or (e) the method results in a positive change in the sleeping pattern of the subject over a defined period of time, wherein the positive change is defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject obtains the total number of hours of sleep recommended by a medical authority for the age group of the subject; and/or (g) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of Parkinson's disease (PD) and/or a related symptom in a patient wherein the PD symptom to be treated, prevented, and/or slowed is hallucination and wherein: (a) the hallucination comprises a visual, auditory, tactile, gustatory or olfactory hallucination; and/or (b) treating the hallucination prevents and/or delays the onset and/or progression of the Parkinson's disease; and/or (c) the method results in a decreased number of hallucinations of the subject over a defined period of time; and/or (d) the method results in a decreased number of hallucinations of the subject over a defined period of time and the decrease in number is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (e) the method results in the subject being hallucination-free; and/or (f) the method results in a decreased severity of hallucinations of the subject over a defined period of time, as measured by one or more medically recognized technique; and/or (g) the method results in a decreased severity of hallucinations of the subject over a defined period of time and the decrease in severity is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as measured by one or more medically recognized technique; and/or (h) the medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or (i) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of Parkinson's disease (PD) and/or a related symptom in a patient wherein the PD symptom to be treated, prevented, and/or slowed is depression and wherein:
(a) treating the depression prevents and/or delays the onset and/or progression of the Parkinson's disease; and/or (b) the method results in improvement in a subject's depression over a defined period of time, as measured by one or more clinically-recognized depression rating scale; and/or (c) the method results in improvement in a subject's depression over a defined period of time, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or (d) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or (e) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of Parkinson's disease (PD) and/or a related symptom in a patient wherein the PD symptom to be treated, prevented, and/or slowed is cognitive impairment, and wherein:

(a) treating the cognitive impairment prevents and/or delays the onset and/or progression of the Parkinson's disease; and/or (b) progression or onset of the cognitive impairment is slowed, halted, or reversed over a defined period of time following administration of the pharmaceutical composition, as measured by a medically-recognized technique; and/or (c) the cognitive impairment is positively impacted by the administration of the pharmaceutical composition, as measured by a medically-recognized technique; and/or (d) the cognitive impairment is positively impacted by the administration of the pharmaceutical composition, as measured by a medically-recognized technique and the positive impact on and/or progression of cognitive decline is measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or (e) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a method of treating, preventing, and/or slowing the onset or progression of Parkinson's disease (PD) and/or a related symptom in a patient wherein the PD symptom to be treated, prevented, and/or slowed is constipation, and wherein: (a) treating the constipation prevents and/or delays the onset and/or progression of the Parkinson's disease; and/or (b) the administration of the pharmaceutical composition causes the subject to have a bowel movement; and/or (c) the method results in an increase in the frequency of bowel movement in the subject over a defined period of time; and/or (d) the method results in an increase in the frequency of bowel movement in the subject and the increase in the frequency of bowel movement is defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (e) as a result of the method the subject has the frequency of bowel movement recommended by a medical authority for the age group of the subject.

The disclosure provides a method of treating, preventing, and/or slowing the onset or progression of Parkinson's disease (PD) and/or a related symptom in a patient wherein the PD symptom to be treated, prevented, and/or slowed is neurodegeneration correlated with PD, and wherein: (a) treating the neurodegeneration prevents and/or delays the onset and/or progression of the Parkinson's disease; and/or (b) the method results in treating, preventing, and/or delaying the progression and/or onset of neurodegeneration in the subject; and/or (c) progression or onset of the neurodegeneration is slowed, halted, or reversed over a defined period of time following administration of the pharmaceutical composition, as measured by a medically-recognized technique; and/or (d) the neurodegeneration is positively impacted by the administration of the pharmaceutical composition, as measured by a medically-recognized technique; and/or (e) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (f) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or (g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

The disclosure provides a pharmaceutical composition comprising at least one active agent selected from the group consisting of: about 0.1% to about 50% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof; about 0.1% to about 50% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof, and combinations thereof, further wherein the pharmaceutical composition comprises: about 1% to about 99% of at least one solvent; about 1% to about 99% of at least surfactant; optionally, about 1% to about 99% of at least one permeation enhancer; and/or optionally, about 30% to about 99% of an adhesive and/or polymer. The disclosure provides a pharmaceutical composition wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, and about 5 μg/mL. The disclosure provides a pharmaceutical composition wherein the active agent is present at a concentration selected from the group consisting of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, and about 50% of the formulation. The disclosure provides a pharmaceutical composition wherein the active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation. The disclosure provides a pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a pharmaceutical composition wherein the pharmaceutical composition comprises one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery. The disclosure provides a pharmaceutical composition wherein the pharmaceutical composition is formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, or transdermal drug-in-adhesive matrix formulation. The disclosure provides a pharmaceutical composition wherein the pharmaceutical composition is formulated as a topical liquid formulation, topical semisolid formulation, topical gel formulation, topical polymer matrix formulation, topical adhesive matrix formulation, topical film forming gel formulation, or topical film forming spray formulation. The disclosure provides a pharmaceutical composition wherein the pharmaceutical composition further comprises carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof. The disclosure provides a pharmaceutical composition wherein the pharmaceutical composition further comprises carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v. The disclosure provides a pharmaceutical composition wherein the pharmaceutical composition is formulated as a transdermal patch. The disclosure provides a pharmaceutical composition wherein the pharmaceutical composition is formulated as a multilayer transdermal matrix system, a metered dose transdermal gel, metered dose transdermal spray, a film forming gel, a film forming spray, or a meter-dose aerosol. The disclosure provides a pharmaceutical composition wherein the pharmaceutical composition is formulated as a topical patch. The disclosure provides a pharmaceutical composition wherein the pharmaceutical composition is formulated as metered dose gel, metered dose spray, gel, cream, solution, emulsion, liquid compositions, semisolid compositions, a matrix of adhesive in combination with polymers, or film forming formulations. The disclosure provides a pharmaceutical composition wherein the pharmaceutical composition is formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof. The disclosure provides a pharmaceutical composition wherein the pharmaceutical composition is formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof. The disclosure provides a pharmaceutical composition wherein the pharmaceutical composition is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a pharmaceutical composition wherein the pharmaceutical composition is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a pharmaceutical composition wherein the pharmaceutical composition is formulated as microneedles. The disclosure provides a pharmaceutical composition wherein the said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route. The disclosure provides a pharmaceutical composition wherein the active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition. The disclosure provides a pharmaceutical composition wherein the pharmaceutical composition is co-administered with at least one additional an active agent selected from the group consisting of: a dopamine precursor, a dopamine receptor agonist, and combinations thereof. The disclosure provides a pharmaceutical composition wherein the pharmaceutical composition is co-administered with at least one additional an active agent selected from the group consisting of: levodopa, bromocriptine, pergolide, pramipexole, cabergoline, ropinorole, apomorphine, and any combination thereof.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION

In Transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week.

Transdermal delivery can reduce the dosing frequency of, for example, cannabidiol and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, alone or in combinations thereof. Through transdermal delivery, transdermal compositions or transdermal formulations or transdermal patch of, for example, cannabidiol and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, alone or in combinations thereof, can be applied topically to skin thereby delivering the drug throughout the duration of topical application. Depending on the requirement, the duration of topical application can be once in a day, once in two days, once in three days, once in four days, once in five days, once in a week. Therefore, transdermal delivery can overcome the multiple dose regimen of oral delivery by reducing the dosing frequency.

Moreover, in transdermal drug delivery the drug is delivered slowly and continuously throughout the duration of topical application hence there are no peaks and troughs in drug plasma concentration which are associated with multiple dose administration in a day. Therefore, by transdermal delivery of cannabidiol and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, alone or in combinations thereof, patients can have the therapeutic effect of the drug for extended period of time without drastic changes in drug plasma concentration.

When a medication is orally administered, its bioavailability generally decreases due to incomplete absorption and first-pass metabolism and may vary from patient to patient. The compositions of the disclosure overcome these problems because in transdermal delivery, active agent is delivered directly into systemic circulation through the skin, and it escapes the first pass hepatic metabolism therefore to achieve the desired therapeutic activity less drug is required, resulting into less adverse effects or side effects. Cannabinol has high lipid solubility and after oral administration undergoes hepatic first pass metabolism, therefore of the administered dose only 10%-20% reaches systemic circulation, thus as compared to oral dose, with transdermal delivery a small dose of cannabidiol can give the desired therapeutic effects at a lower dose than oral.

Transdermal delivery is easy, noninvasive and convenient. Administration of a transdermal patch or transdermal composition does not require medical supervision as patients can topically apply the transdermal patch or transdermal composition themselves.

Moreover, in case of any adverse effect, side effect or emergency transdermal delivery gives the liberty to terminate the therapy anytime by taking off the transdermal patch or transdermal composition from skin.

As used herein the term “active pharmaceutical ingredient” (“API”) or “pharmaceutically active agent” is a drug or agent which can be employed as disclosed herein and is intended to be used in the human or animal body in order to heal, to alleviate, to prevent or to diagnose diseases, ailments, physical damage or pathological symptoms; allow the state, the condition or the functions of the body or mental states to be identified; to replace active substances produced by the human or animal body, or body fluids; to defend against, to eliminate or to render innocuous pathogens, parasites or exogenous substances or to influence the state, the condition or the functions of the body or mental states. Drugs in use can be found in reference works such as, for example, the Rote Liste or the Merck Index. Examples which may be mentioned include, for example, CBD and/or THC.

As used herein, the term “agent” refers to any molecule, compound, methodology and/or substance for use in the prevention, treatment, management and/or diagnosis of a disease or condition.

As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes, for example, chemical modification of a compound of the disclosure, or extracted from botanical sources or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably.

As used herein, the term “transdermal delivery” means delivery of drug into systemic circulation through the skin.

As used herein, “pharmaceutically acceptable salts” refer to derivatives of the disclosed compounds wherein the therapeutic compound is modified by making acid or base salts thereof. Examples of pharmaceutically acceptable salts include, but are not limited to, mineral or organic acid salts of the active agent. The pharmaceutically acceptable salts include the conventional non-toxic salts, for example, from non-toxic inorganic or organic acids. For example, such conventional non-toxic salts include those derived from inorganic acids such as hydrochloric, hydrobromic, sulfuric, sulfonic, sulfamic, phosphoric, nitric and the like; and the salts prepared from organic acids such as amino acids, acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and other known to those of ordinary skill in the pharmaceutical sciences. Lists of suitable salts are found in texts such as Remington's Pharmaceutical Sciences, 18th Ed. (Alfonso R. Gennaro, ed.; Mack Publishing Company, Easton, Pa., 1990); Remington: the Science and Practice of Pharmacy 19^{th} Ed. (Lippincott, Williams & Wilkins, 1995); Handbook of Pharmaceutical Excipients, 3^{rd} Ed. (Arthur H. Kibbe, ed.; Amer. Pharmaceutical Assoc., 1999); the Pharmaceutical Codex: Principles and Practice of Pharmaceutics 12^{th} Ed. (Walter Lund ed.; Pharmaceutical Press, London, 1994); The United States Pharmacopeia: The National Formulary (United States Pharmacopeial Convention); and Goodman and Gilman's: the Pharmacological Basis of Therapeutics (Louis S. Goodman and Lee E. Limbird, eds.; McGraw Hill, 1992), the disclosures of which are hereby incorporated by reference.

An amount is “effective” as used herein, when the amount provides an effect in the subject. As used herein, the term “effective amount” means an amount of a compound or composition sufficient to significantly induce a positive benefit, including independently or in combinations the benefits disclosed herein, but low enough to avoid serious side effects, i.e., to provide a reasonable benefit to risk ratio, within the scope of sound judgment of the skilled artisan. For those skilled in the art, the effective amount, as well as dosage and frequency of administration, may be determined according to their knowledge and standard methodology of merely routine experimentation based on the present disclosure.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is an elderly human. In another embodiment, the subject is a human adult. In another embodiment, the subject is a human child. In yet another embodiment, the subject is a human infant.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the terms “prevent,” “preventing” and “prevention” in the context of the administration of a therapy to a subject refer to the prevention or inhibition of the recurrence, onset, and/or development of a disease or condition, or a combination of therapies (e.g., a combination of prophylactic or therapeutic agents).

As used herein, the terms “therapies” and “therapy” can refer to any method(s), composition(s), and/or agent(s) that can be used in the prevention, treatment and/or management of a disease or condition, or one or more symptoms thereof.

As used herein, the terms “treat,” “treatment,” and “treating” in the context of the administration of a therapy to a subject refer to the reduction or inhibition of the progression and/or duration of a disease or condition, the reduction or amelioration of the severity of a disease or condition, and/or the amelioration of one or more symptoms thereof resulting from the administration of one or more therapies.

As used herein, the term “about” when used in conjunction with a stated numerical value or range has the meaning reasonably ascribed to it by a person skilled in the art, i.e., denoting somewhat more or somewhat less than the stated value or range.

### Active Agent

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. The disclosure provides for, for example, transdermal formulations and/or topical formulations comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol IUPAC Name 2-[(1R,6R)-6-isopropenyl-3-methylcyclohex-2-en-1-yl]-5-pentylbenzene-1,3-diol Chemical Formula: C₂₁H₃₀O₂ Molecular weight: 314.46 dalton Chemical structure is shown below as formula I

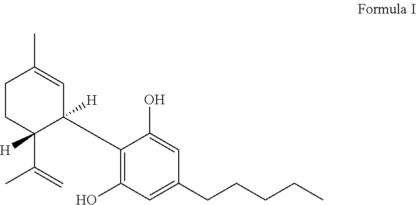 [SMILES: C=C(C)[C@@H]1CCC(C)=C[C@H]1c1c(O)cc(CCCCC)cc1O]

Tetrahydrocannabinol (THC) IUPAC Name (−)-(6aR,10aR)-6,6,9-Trimethyl-3-pentyl-6a,7,8,10a-tetrahydro-6H-benzo[c]chromen-1-ol Chemical Formula: C₂₁H₃₀O₂ Molecular weight: 314.47 dalton.

Chemical structure is shown below as formula II

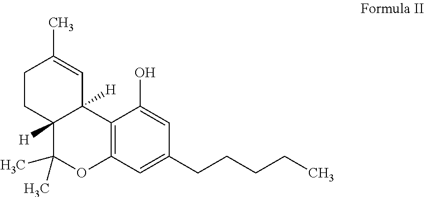 [SMILES: CCCCCc1cc(O)c2c(c1)OC(C)(C)[C@@H]1CCC(C)=C[C@@H]21]

As used herein, the word cannabis refers to all pharmaceutically acceptable forms of cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form or ion-pairs. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer or its ion-pairs. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, solid solution thereof, polymorph thereof, stereoisomers thereof, powder form thereof, liquid form thereof, ion-pairs thereof, solution of cannabidiol in solvents such as but not limited to methanol, etc. alone or in combinations thereof.

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, solid solution thereof, powder form thereof, liquid form thereof, ion-pairs thereof, polymorph thereof, stereoisomers thereof, solution of THC in solvents such as but not limited to methanol, heptane, etc. alone or in combinations thereof.

As used herein, synthetic cannabinoids include at least the following:

AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374—palmitylsulfonyl fluoride; AM-381—stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl)indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl)indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl)indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54× selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl)indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-1-oyl)indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator-activated receptor α (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28× more strongly to CB2 than CB1. and has been shown to have immunomodulatory effects, and may be useful in the treatment of pain and inflammation; JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051—an analgesic with a high affinity for the CB1 receptor, but is a much stronger agonist for CB2, JWH-057—a 1-deoxy analog of Δ8-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173-fold selective CB2 agonist; JWH-122—a potent and fairly selective CB1 agonist; JWH-133—a potent and highly selective CB2 receptor agonist; 1JWH-139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175—(1-pentylindol-3-yl)naphthalen-1-ylmethane, 22 nM at CB1, JWH-176—1-([(1E)-3-pentylinden-1-ylidine]methyl)naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl)methane; JWH-185—1-pentyl-1H-indol-3-yl-(4-methoxy-1-naphthyl)methane; JWH-192—(1-(2-morpholin-4-ylethyl)indol-3-yl)-4-methylnaphthalen-1-ylmethane; JWH-193—(1-(2-morpholin-4-ylethyl)indol-3-yl)-4-methylnaphthalen-1-ylmethanone; JWH-194—2-methyl-1-pentyl-1H-indol-3-yl-(4-methyl-1-naphthyl)methane; JWH-195—(1-(2-morpholin-4-ylethyl)indol-3-yl)-naphthalen-1-ylmethane; JWH-196—2-methyl-3-(1-naphthalenylmethyl)-1-pentyl-1H-Indole; JWH-197—2-methyl-1-pentyl-1H-indol-3-yl-(4-methoxy-1-naphthyl)methane; JWH-198—(1-(2-morpholin-4-ylethyl)indol-3-yl)-4-methoxynaphthalen-1-ylmethanone; JWH-199—(1-(2-morpholin-4-ylethyl)indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic chemical from the naphthoylindole family, which acts as a potent cannabinoid agonist at both the CB1 and CB2 receptors; JWH-213—a potent and fairly selective CB2 agonist; JWH-229—1-methoxy-3-(1′,1′-dimethylhexyl)-Δ8-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl)indole); JWH-258—a potent and mildly selective CB1 agonist; JWH-302—(1-pentyl-3-(3-methoxyphenylacetyl)indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2receptor affinity with little affinity for the CB1 receptor; JWH-359—a dibenzopyran cannabinoid that is a potent and selective CB2 receptor agonist; JWH-387—1-pentyl-3-(4-bromo-1-naphthoyl)indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E. coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for the transdermal and/or topical compositions as disclosed herein comprising, for example, biosynthetic CBD, alone or in combination with other active agents.

According to certain embodiments, transdermal and/or topical compositions described herein are for the compositions and methods for the treatment and/or prevention and/or control of Parkinson's Disease in a patient comprising administration of, for example, CBD and/or THC, as set forth herein. According to certain embodiments, transdermal and/or topical compositions described herein are for the reduction in severity of Parkinson's disease.

According to certain embodiments described herein, a pharmaceutical composition or transdermal formulation or topical formulation contains cannabidiol and/or THC—the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, polymorphs thereof, stereoisomers thereof, coated form thereof, solid solution thereof, ion-pairs thereof, solution thereof in solvents alone or in combinations thereof. More preferably transdermal and topical formulation may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, naturally occurring forms thereof, active metabolites thereof, polymorphs thereof, ion-pairs thereof, stereoisomers thereof, coated form thereof, solution of cannabidiol in methanol, alone or in combinations thereof. More preferably transdermal and topical formulation may include THC, the free base thereof, salts thereof, isomers thereof, amorphous forms thereof, crystalline forms thereof, co crystalline forms thereof, prodrugs thereof, analogs thereof, derivatives thereof, ion-pairs thereof, synthetic forms thereof, biosynthetic forms thereof, naturally occurring forms thereof, active metabolites thereof, polymorphs thereof, stereoisomers thereof, coated form thereof, solution of cannabidiol in methanol, alone or in combinations thereof.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites polymorphs thereof, stereoisomers thereof, coated form thereof, ion-pairs thereof. For example, the active agent's free base or its salts or its isomers or its amorphous form or its crystalline form or its co crystalline form or its solid solution or its prodrugs or its analogs or its derivatives or synthetic forms polymorphs thereof, ion-pairs thereof, stereoisomers thereof, coated form thereof. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this invention are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

As used herein, the term active agent includes the free base thereof, salts thereof, isomers thereof, amorphous forms thereof, crystalline forms thereof, co crystalline forms thereof, prodrugs thereof, analogs thereof, derivatives thereof, synthetic forms thereof, polymorphs thereof, stereoisomers thereof, coated form thereof, ion-pairs thereof, alone or in combinations thereof. In certain embodiments the active agent is highly purified. In certain embodiments the active agent is a highly pure synthetic. In certain embodiments the active agent is a highly purified extract of active agent which comprises a concentration equal to or greater than about 90%, 91%, 92%, 93%, 94%, 95%, 96%, 97%, 98%, 99%, 99.5%, or 99.75% (w/w).

In certain embodiments the active agent is provided at a concentration equal to or greater than about 90%, 91%, 92%, 93%, 94%, 95%, 96%, 97%, 98%, 99%, 99.5%, 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, or 45 mg/kg/day. In certain embodiments, the active agent is titrated from 5 to 20-25 mg/kg/day and optionally maintained for 10-15 days. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg. In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% of the formulation. In exemplary embodiments, formulations of the disclosure may comprise active agent at a concentration of about 0.1 to about 50%, about 1 to 20%, of about 5% to 25%, about 10% to about 20%, or about 15% to about 18%, about 30% to about 70%, about 35% to about 65%, about 63.13%, about 40% to about 64% w/w, about 95 to about 98%, or about 95 to about 97%. In exemplary formulations of the disclosure, the active agent will represent approximately about 1% to about 10%, about 1% to about 9%, about 1% to about 8%, about 1% to about 7%, about 1% to about 6%, about 1% to about 5%, about 0.1 wt % to about 50% wt %, 1 wt % to 75 wt %, preferably 2 wt % to 30 wt %, more preferably 5 wt. % to 20 wt. % of the formulation.

The present disclosure provides compositions and methods that have the unique benefit of providing treatment of a condition as set forth herein that will allow a patient or heath care provider to titrate dose to the most effective dose per patient with the use, such as the addition, of an incremental dose patch.

The present disclosure provides compositions and methods which have the unique ability to immediately stop drug administration by simply removing the patch. For example, a patient may start with a dose that has the onset of unwanted negative side effects. The patient or heath care provider can control or stop administration (e.g., remove the patch, or titrate the administration with a lower dose patch) before it gets worse, and also dramatically shorten the time of the unwanted experience.

As used herein, the term “pharmaceutically acceptable salts” includes acid addition salts or addition salts of free bases. The term “pharmaceutically acceptable salts” of the active agent within its scope all the possible isomers and their mixtures, and any pharmaceutically acceptable metabolite, bioprecursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being. As used herein, the term “transdermal delivery” means delivery of drug into systemic circulation through the skin.

### Indications

Therapeutic indications include a variety of central nervous system disorders characterized by motor and/or non-motor disturbances that can be seen in neurodegenerative diseases such as but not limited to Parkinson's disease (PD), Restless leg syndrome, Huntington's disease, and Alzheimer's disease but also neuropsychiatric diseases such as, but not limited to schizophrenia, attention deficit hyperactivity disorder and drug addiction. The compositions and methods of the present disclosure are intended for treatment of neurodegenerative diseases and disorders such as Parkinson's disease and/or other conditions for which treatment with a dopamine agonist is therapeutically beneficial.

In addition to neurodegenerative diseases and disorders, other conditions in which an increase in dopaminergic turnover may be beneficial are in the improvement of mental functions including various aspects of cognition. It may also have a positive effect in depressed patients, and it may also be used in the treatment of obesity as an anorectic agent and in the treatment of drug addiction. It may improve minimal brain dysfunction (MBD), narcolepsy, attention deficit hyperactivity disorder and potentially the negative, the positive as well as the cognitive symptoms of schizophrenia.

Restless leg syndrome (RLS) and periodic limb movement disorder (PLMD) are alternative indications, which are clinically treated with dopamine agonists. In addition, impotence, erectile dysfunction, SSRI induced sexual dysfunction, ovarian hyperstimulation syndrome (OHSS) and certain pituitary tumors (prolactinoma) are also likely to be improved by treatment with dopamine agonists. Dopamine is involved in regulation of the cardiovascular and renal systems, and accordingly, renal failure and hypertension can be considered alternative indications for the compositions and methods as disclosed herein.

The compositions and methods of the disclosure may be used for treatments of any suitable condition where one or more dopaminergic agents are used that have side effects. Examples of conditions include, but are not limited to, Parkinson's disease, Alzheimer, dopa-responsive dystonia, cerebral palsy, postischemic contractile dysfunction, severe ovarian hyperstimulation syndrome, pediatric movement disorders and non-oliguric renal failure.

In various embodiments, the compositions and methods of treatment are directed to direct symptoms of Parkinson's Disease rather than treatment of a dopaminergic agent-induced side effect. For example, in various embodiments, the methods of treatment are directed to treatment of gait and balance deficits resulting directly from Parkinson's Disease. In various embodiments, nicotine is administered separately from any dopaminergic agent. In various embodiments, the subject undergoing treatment with nicotine is not receiving a dopaminergic agent. In various embodiments, the subject undergoing treatment with nicotine is not receiving levodopa, carbidopa, or combinations thereof.

In various embodiments, the compositions and methods of treatment are directed to include treatment, prevention or slowing of Parkinson's Disease Dementia (PDD). In other embodiments, the present disclosure provides for a dosage form for treatment of gait and balance problems in Parkinson's Disease (PD), comprising topical administration of THC and/or CBD, as disclosed herein. In various embodiments for direct treatment of symptoms of Parkinson's Disease, a dopaminergic agent may be included or excluded from the method of treatment. In various embodiments, levodopa and/or carbidopa may be included or excluded from the methods herein.

In some embodiments, the invention provides compositions and methods utilizing THC and/or CBD, as disclosed herein to reduce, alleviate, or eliminate symptoms of Parkinson's Disease or symptoms associated with Parkinson's Disease, e.g., a side effect associated with dopaminergic agent treatment. In some embodiments, the invention provides compositions and methods utilizing THC and/or CBD, as disclosed herein, e.g., to reduce or eliminate a side effect associated with dopaminergic agent treatment. In some embodiments, the THC and/or CBD, as disclosed herein reduces or eliminates a side effect associated with dopaminergic agent treatment. Dopaminergic agents include a dopamine precursor or a dopamine receptor agonist. Examples of dopaminergic agents include levodopa, bromocriptine, pergolide, pramipexole, cabergoline, ropinorole, apomorphine, or a combination thereof.

Non-limiting exemplary PD symptoms include but are not limited to (a) at least one non-motor aspect of experiences of daily living as defined by Part I of the Unified Parkinson's Disease Rating Scale (UPDRS) selected from the group consisting of cognitive impairment, hallucinations and psychosis, depressed mood, anxious mood, levodopa induced nausea, apathy, features of dopamine dysregulation syndrome, sleep problems, daytime sleepiness, pain, urinary problems, constipation problems, lightheadedness on standing, and fatigue; (b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing; (c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor; (d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia; (e) constipation; (f) depression; (g) cognitive impairment; (h) short or long term memory impairment; (i) concentration impairment; (j) coordination impairment; (k) mobility impairment; (l) speech impairment; (m) mental confusion; (n) sleep problem, sleep disorder, or sleep disturbance; (o) circadian rhythm dysfunction; (p) hallucinations; (q) fatigue; (r) REM disturbed sleep; (s) REM behavior disorder; (t) erectile dysfunction; (u) postural hypotension; (v) correction of blood pressure or orthostatic hypotension; (w) nocturnal hypertension; (x) regulation of temperature; (y) improvement in breathing or apnea; (z) correction of cardiac conduction defect; (aa) amelioration of pain; (bb) urinary incontinence, or restoration of bladder sensation and urination; (cc) mood swings; (dd) apathy; (ee) control of nocturia; (ff) neurodegeneration; (gg) anxiety; including anxiety related to public speaking; (hh) improving speaking ability, including public speaking ability; and/or (ii) Parkinson's Disease Dementia. In some embodiments, (a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof, (b) the REM-behavior disorder comprises vivid dreams, nightmares, and acting out the dreams by speaking or screaming, or fidgeting or thrashing of arms or legs during sleep; or (c) the hallucination comprises a visual, auditory, tactile, gustatory or olfactory hallucination.

In embodiments where the PD symptom to be treated, prevented, and/or slowed is a sleep problem, sleep disorder, sleep disturbance, circadian rhythm dysfunction, REM disturbed sleep, or REM behavior disorder, (a) treating the sleep problem, sleep disorder, sleep disturbance may prevent or delay the onset and/or progression of the PD; (b) the sleep problem, sleep disorder or sleep disturbance may comprise a delay in sleep onset, sleep fragmentation, REM-behavior disorder, sleep-disordered breathing including snoring and apnea, day-time sleepiness, micro-sleep episodes, narcolepsy, hallucinations, or any combination thereof; (c) the REM-behavior disorder may comprise vivid dreams, nightmares, and acting out the dreams by speaking or screaming, or fidgeting or thrashing of arms or legs during sleep; (d) the method may result in a positive change in the sleeping pattern of the subject; (e) the method may result in a positive change in the sleeping pattern of the subject, wherein the positive change can be defined as: (i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or (f) as a result of the method the subject may obtain the total number of hours of sleep recommended by a medical authority for the age group of the subject.

In embodiments where the PD symptom to be treated, prevented, and/or slowed is hallucinations, (a) the hallucination may comprise a visual, auditory, tactile, gustatory or olfactory hallucination; (b) treating the hallucination may prevent and/or delay the onset and/or progression of the Parkinson's disease; (c) the method results in a decreased number or severity of hallucinations of the subject; (d) the method may result in a decreased number or severity of hallucinations of the subject and the decrease in number or severity in hallucinations can be defined as a reduction in occurrences or severity of hallucinations selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (e) the method may result in the subject being hallucination-free.

In embodiments where the PD symptom to be treated, prevented, and/or slowed is depression, (a) treating the depression may prevent and/or delay the onset and/or progression of the Parkinson's disease; (b) the method may result in improvement in a subject's depression, as measured by one or more clinically-recognized depression rating scale; (c) the method may result in improvement in a subject's depression, as measured by one or more clinically-recognized depression rating scale and the improvement can be in one or more depression characteristics selected from the group consisting of mood, behavior, bodily functions such as eating, sleeping, energy, and sexual activity, and/or episodes of sadness or apathy; and/or (d) the method may result in improvement in a subject's depression, as measured by one or more clinically-recognized depression rating scale, and the improvement a subject experiences following treatment can be about 5, about 10, about 15, about 20, about 25, about 30, about 35, about 40, about 45, about 50, about 55, about 60, about 65, about 70, about 75, about 80, about 85, about 90, about 95 or about 100%.

In embodiments where the PD symptom to be treated, prevented, and/or slowed is cognitive impairment, (a) treating the cognitive impairment may prevent and/or delay the onset and/or progression of the Parkinson's disease; (b) progression or onset of the cognitive impairment can be slowed, halted, or reversed over a defined period of time following administration of the administration of the pharmaceutical composition, as measured by a medically-recognized technique; and/or (c) the cognitive impairment can be positively impacted by the administration of the pharmaceutical composition, as measured by a medically-recognized technique; (d) the cognitive impairment can be positively impacted by the fixed escalated dose of the administration of the pharmaceutical composition, as measured by a medically-recognized technique and the positive impact on and/or progression of cognitive decline can be measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or (e) the progression or onset of cognitive impairment can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

In embodiments where the PD symptom to be treated, prevented, and/or slowed is constipation, (a) treating the constipation may prevent and/or delay the onset and/or progression of the Parkinson's disease; (b) the administration of the pharmaceutical composition may cause the subject to have a bowel movement; (c) the method may result in an increase in the frequency of bowel movement in the subject; (d) the method may result in an increase in the frequency of bowel movement in the subject and the increase in the frequency of bowel movement can be defined as: (i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or (ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; (e) as a result of the method the subject may have the frequency of bowel movement recommended by a medical authority for the age group of the subject.

In embodiments where the PD symptom to be treated, prevented, and/or slowed is neurodegeneration correlated with PD, (a) treating the neurodegeneration may prevent and/or delay the onset and/or progression of the Parkinson's disease; (b) the method may result in treating, preventing, and/or delaying the progression and/or onset of neurodegeneration in the subject; (c) progression or onset of the neurodegeneration can be slowed, halted, or reversed over a defined period of time following administration of the administration of the pharmaceutical composition, as measured by a medically-recognized technique; and/or (d) the neurodegeneration can be positively impacted by the administration of the pharmaceutical composition, as measured by a medically-recognized technique. In further embodiments, (a) the positive impact and/or progression of neurodegeneration can be measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or (b) the progression or onset of neurodegeneration can be slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique.

The disclosure encompasses use of the compounds and methods as disclosed herein for treatment of the diseases, symptoms, and disorders as disclosed herein.

### Additional Active Agents

As used herein the term “combination administration” of a compound, therapeutic agent or known drug with the combination of the present invention means administration of the drug and the one or more compounds at such time that both the known drug and/or combination will have a therapeutic effect. In some cases, this therapeutic effect will be synergistic. Such concomitant administration can involve concurrent (i.e., at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

Further, active ingredient(s), where applicable, may be present either in the form of one substantially optically pure enantiomer or as a mixture of enantiomers or polymorphs thereof.

The active ingredient(s) may comprise one or more of the following therapeutic classes but not limited to dopaminergic agents.

In one aspect, the invention provides compositions and methods to reduce or eliminate the effects of a dopaminergic agent. In some embodiments, the compositions and methods retain or enhance a desired effect of the dopaminergic agent, e.g., antiparkinsonian effect. The methods and compositions of the invention apply to any dopaminergic agent for which it is desired to reduce one or more side effects. In some embodiments, the compositions and methods of the invention utilize a dopamine precursor. In some embodiments, the compositions and methods of the invention utilize a dopamine agonist. In some embodiments, the dopaminergic agent is levodopa, bromocriptine, pergolide, pramipexole, cabergoline, ropinorole, apomorphine, or a combination thereof. In some embodiments, the dopaminergic agent is levodopa. In some embodiments, the compositions and methods of the disclosure utilize one or more agents used in the art in combination with a dopamine agent treatment to achieve a therapeutic effect. For instance, in one exemplary embodiment the compositions and methods of the invention utilize levodopa in combination with an agent such as carbidopa, which blocks the conversion of levodopa to dopamine in the blood. In another exemplary embodiment, the compositions and methods of the invention utilize levodopa in combination with a COMT Inhibitor, such as entacapone. In another exemplary embodiment, the compositions and methods of the invention utilize levodopa in combination with a monoamine oxidase type B (MAO-B) inhibitor such as selegiline. In yet another exemplary embodiment, the compositions and methods of the invention utilize levodopa in combination with amantadine.

Levodopa, an aromatic amino acid, is a white, crystalline compound, slightly soluble in water, with a molecular weight of 197.2. It is designated L-3,4-dihydroxyphenylalanine (S)-2-amino-3-(3,4-dihydroxyphenyl)propanoic acid. Its structural formula is

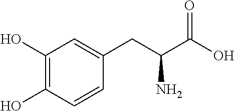 [SMILES: N[C@@H](Cc1ccc(O)c(O)c1)C(=O)O]

Levodopa is used for the treatment of Parkinson's disease. Parkinson's disease is a progressive, neurodegenerative disorder of the extrapyramidal nervous system affecting the mobility and control of the skeletal muscular system. Its characteristic features include resting tremor, rigidity, and bradykinetic movements. Current evidence indicates that symptoms of Parkinson's disease are related to depletion of dopamine in the corpus striatum. Administration of dopamine is ineffective in the treatment of Parkinson's disease apparently because it does not cross the blood-brain barrier. However, levodopa, the metabolic precursor of dopamine, does cross the blood-brain barrier, and presumably is converted to dopamine in the brain. This is thought to be the mechanism whereby levodopa relieves symptoms of Parkinson's disease.

However, although initially very effective, long term treatment with levodopa gives rise to multiple complications. Levodopa treatment may cause nausea, vomiting, involuntary movements (e.g. dyskinesias), mental disturbances, depression, syncope, and hallucinations.

### Side Effects

The principal adverse reactions of dopaminergic agent include headache, diarrhea, hypertension, nausea, vomiting, involuntary movements (e.g., dyskinesias), mental disturbances, depression, syncope, hallucinations, and abnormal renal function.

The disclosure provides compositions and methods utilizing THC and/or CBD, as disclosed herein that reduces or eliminates a side effect associated with dopaminergic agent treatment. In some embodiments, the disclosure provides compositions and methods utilizing THC and/or CBD, as disclosed herein, that reduces or eliminates dyskinesias associated with dopaminergic agent treatment.

### Pharmaceutical Compositions

According to certain embodiments described herein, pharmaceutical composition or transdermal formulation and/or topical formulation of contains active agents such as cannabinoids, and derivatives of these compounds. More preferably transdermal and/or topical formulation may include active agents such as CBD and/or THC, and derivatives of these compounds.

One embodiment of the present disclosure can be a transdermal drug delivery system which may include without any limitation to transdermal formulation, transdermal patches, microneedles, iontophoresis, metered dose transdermal spray, metered dose transdermal gel, transdermal aerosols, transdermal film forming formulations.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch, metered dose transdermal system, sachet, etc. Transdermal formulations which includes matrix patches without any limitations like adhesive matrix patch, drug in adhesive matrix patch, non-adhesive matrix patch, a transdermal matrix formulation as drug in adhesive matrix patch is preferred. Other transdermal formulations include transdermal gel, transdermal meter dose spray, transdermal meter dose aerosols, transdermal film forming formulation, microneedles.

Without any limitation, transdermal patch may include all transdermal drug delivery systems stated in art preferably but not limited to a multilayer transdermal matrix system, reservoir patch, matrix patch, bilayer matrix patch, multilayer matrix patch, microreservoir patch, adhesive systems, transdermally applicable tape and other.

In certain embodiments of the present disclosure, a transdermal patch comprises transdermal formulation containing active agents such as CBD and/or THC, and derivatives of these compounds contained in a reservoir or a matrix, and an adhesive which allows the transdermal patch to adhere to the skin, allowing the passage of the active agents such as CBD and/or THC, and derivatives of these compounds from the transdermal patch through the skin of the patient. The transdermal delivery system can be occlusive, semi-occlusive or non-occlusive, and can be adhesive or non-adhesive.

The transdermal formulation comprising active agents such as CBD and/or THC, and derivatives of these compounds can be incorporated within the patch and patch can be applied topically to the skin surface. The patch can be left on the subject for any suitable period of time.

In some embodiments, the transdermal patches provide for a constant rate of delivery of the active components of the transdermal patch over a predetermined time period. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a steady absorption rate of the active components of the transdermal patches by the patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a constant blood serum level of the active components of the transdermal patches in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein allow for reduced variability in dosage of active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical compositions described herein provide a plasma concentration of the active agent components of the transdermal patches in a therapeutic range in a patient over a predetermined time. In exemplary embodiments as disclosed herein, the pharmaceutical composition provides a blood serum level of active agent selected from without any limitation, of, for example, about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, about 5 μg/mL, and ranges thereof. In one aspect, the pharmaceutical composition provides a blood serum level of active agent in the range of 0.01 ng/mL-400 ng/mL. In another aspect, transdermal patch provides a blood serum level of active agent in the range of 0.01 ng/mL-100 ng/mL. In yet another aspect the pharmaceutical composition provides a blood serum level of active agent in the range of from 0.01-1 ng/ml to 1-100 ng/ml to 100-500 ng/ml to 500-1000 ng/ml to 1000-5000 ng/ml.

In yet further embodiments, the pharmaceutical compositions described herein provide a plasma concentration of the active agent components of the transdermal patches in a therapeutic range in a patient over a predetermined time. In exemplary embodiments as disclosed herein, the pharmaceutical composition provides a blood serum level of active agent selected from without any limitation, of, for example, about 0.01 microgm/mL, about 0.02 microgm/mL, about 0.05 microgm/mL, about 0.1 microgm/mL, about 0.2 microgm/mL, about 0.5 microgm/mL, about 1 microgm/mL, about 2 microgm/mL, about 5 microgm/mL, about 10 microgm/mL, about 20 microgm/mL, about 50 microgm/mL, about 100 microgm/mL, about 200 microgm/mL, about 500 microgm/mL, about 1 milligm/mL, about 2 milligm/mL, about 5 milligm/mL, and ranges thereof. In one aspect, the pharmaceutical composition provides a blood serum level of active agent in the range of 0.01 microgm/mL-400 microgm/mL. In another aspect, the pharmaceutical composition provides a blood serum level of active agent in the range of 0.01 microgm/mL-100 microgm/mL. In yet another aspect the pharmaceutical composition provides a blood serum level of active agent in the range of from 0.01-1 microgm/mL to 1-100 microgm/mL to 100-500 microgm/mL to 500-1000 microgm/mL to 1000-5000 microgm/mL.

The topical formulation stated in the art which include, for example without any limitation, semisolids such as ointment, cream, emulsion, micro emulsion, nano emulsion, paste, balms, gels, lotions, mousses. Liquids such as solutions, suspensions, micro suspension, nano suspension, dispersions, nano dispersion etc. Sprays, aerosols, magma, etc. The topical formulation comprising such as CBD and/or THC, and derivatives of these compounds can be topically applied to the skin surface for topical delivery of such CBD and/or THC, and derivatives of these compounds.

One embodiment of the present disclosure can be a topical drug delivery system which may include without any limitation to topical patches, topical formulation, metered dose topical spray, topical film forming formulation, topical drug-in-adhesive patches, topical matrix patches, topical aerosols, metered dose topical gel.

Multilayer Polymer Drug Matrix System: For instance, a transdermal drug delivery system is contemplated where the active substance matrix is constructed using water soluble polymers, which is then coated on the adhesive layer. Further, the active substance reservoir can be prepared as a polymer matrix. In addition, the active substance reservoir can be confined on the skin facing side of the transdermal drug delivery system by an active substance permeable membrane and on the opposite side from the skin by an active substance impermeable layer followed by adhesive layer.

The disclosure provides a transdermal drug delivery system comprising an active substance matrix containing area is a double or multilayered active substance matrix. In another embodiment, the active substance, CBD/PSI is in the simplest case dispersed, coarsely, colloidally or molecularly, in a solution or melt of base polymers. In the further a transdermal drug delivery system manufacturing techniques, the CBD/PSI is in the form of supersaturated solution, nano-emulsion or nano-suspension, amorphous, crystalline, co-crystals, coated with base polymers or solubilize in polymers using hot melt extrusion process.

The disclosure also includes such embodiments where the CBD/PSI matrix has a two or multi-layered structure, also called multi-laminate drug in adhesive patch. For example, the various matrix layers may contain polymer constitutes from the above-mentioned polymers. In this case, the matrix layers are differing from each other's in the term of polymer or pressure sensitive or hot melt polymers composition, CBD/PSI concentration, different permeating enhancers or solubilizers. The layers can be separated using semi-permeable membrane between two distinct drug-in-adhesive layers or multiple drug-in-adhesive layers under a single backing film. The term polymer film includes polymer without any limitation pressure sensitive adhesive and/or non-adhesive polymer.

In one aspect the disclosure further provides a polymer matrix formulation comprising CBD/PSI and a polymeric vehicle system. The vehicle system can include solvents (e.g., a solubilizer), permeability enhancing excipients and polymer or gelling agent or thickening agent, if required acid or base for pH adjustment.

Pretreatment: Various approaches have been used to open the barrier property of stratum corneum for drug permeation enhancement. Pretreatment with the use of chemical penetration enhancers is one of the techniques employed. The pretreatment has a potential to modulate the outermost layer of the skin reversibly and facilitate the drug uptake. Penetration enhancers act on lipid and protein regions in combination or alone on each region.

Penetration enhancers may be incorporated into the formulations described herein (e.g., transdermal drug delivery systems including drug in adhesive layers and separate adhesive and drug containing layers), however, it can lead to some incompatibility or interactions within the ingredients. Therefore, the present disclosure provides the alternative method of skin penetration enhancement as to preparation/pretreatment the skin with some penetration enhancers or a combination of penetration enhancers before the patch application.

Pretreatment applications described herein include application of a gel/spray/solution/wetting agent/soaked swab/soaked cotton ball/soaked gauzes to the skin prior to application of drug containing product, intended to be a patch. However, it is to be understood that the pretreatment composition can include another topical dosage form, solution gel, cream, etc. For instance, the pretreatment composition can be its own individual patch, such as CuradMediplast, a 40% salicylic acid patch, or a placebo patch comprising non-volatile components such as acrylic, silicone, or PIB adhesives or combinations thereof with an optional addition of a skin permeation enhancers to promote delivery of an active pharmaceutical ingredient through the skin.

The present disclosure provides a pretreatment composition wherein the penetration enhancers are incorporated in the form the topical dosage form as solution, gel, cream, spray, wetting agent, soaked cotton balls and gauzes. In yet another embodiment pretreatment composition preferably but not limited to gel can be incorporated in a reservoir patch.

Topical formulation which includes liquids for example without any limitation like solutions, suspensions, dispersions, emulsion. Topical formulation includes semisolids for example without any limitations like gels, ointments, emulsions, creams, suspension, paste, lotion, balm. Liquid formulation and/or gel formulation incorporated in without any limitation to topical patch, metered dose topical system, sachet, etc. Topical formulations which includes polymer matrix patch without any limitations like adhesive matrix patch, non-adhesive matrix, drug-in-adhesive matrix patch, a topical matrix formulation as drug in adhesive matrix patch is preferred. Other topical formulations include such as but not limited to topical gel, metered dose topical spray, metered dose topical aerosols, topical film forming formulation.

Without any limitation, topical patch may include all topical drug delivery systems stated in art preferably but not limited to a multilayer transdermal matrix system, reservoir patch, matrix patch, bilayer matrix patch, multilayer matrix patch, microreservoir patch, adhesive systems, topically applicable tape and other.

In certain embodiments of the present disclosure, a topical patch comprises topical formulation containing active agents such as diclofenac and/or CBD and/or THC, and derivatives of these compounds contained in a reservoir or a matrix, and an adhesive which allows the topical patch to adhere to the skin, allowing the passage of the active agents such as diclofenac and/or CBD and/or THC, and derivatives of these compounds from the topical patch to the skin of the patient. The topical delivery system can be occlusive, semi-occlusive or non-occlusive, and can be adhesive or non-adhesive.

The topical formulation comprising active agents such as diclofenac and/or CBD and/or THC, and derivatives of these compounds can be incorporated within the patch and patch can be applied topically to the skin surface. The patch can be left on the subject for any suitable period of time.

The transdermal formulation and/or topical formulation of some embodiments of the present disclosure may include carriers or ingredients in effective amount either alone or in combinations thereof without any limitation to the following carriers or ingredients such as solvents, gelling agents, polymers, pressure sensitive adhesive polymers, adhesive polymers biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, bulking agents, excipients, material to prepare patch, material to prepare matrix patch, material to prepare reservoir patch etc.

Active agents may be dissolved, suspended, dispersed or uniformly mixed in the above stated single carrier, mixture of carriers and combinations of carrier. Any combination of two or more drugs such as such as CBD and/or THC, and derivatives of these compounds may be dissolved, suspended, dispersed or uniformly mixed in the above stated single carrier, mixture of carriers and combinations of carrier.

The desired optimum transdermal and/or topical formulation of such as CBD and/or THC, and derivatives of these compounds alone or in combinations thereof may comprise without any limitation to following carriers as stated from example 1 to example 12 either alone or in combinations thereof. According to certain embodiments, transdermal and/or topical compositions described herein are for the treatment and/or prevention and/or control of, for example, Parkinson's Disease and symptoms associated with Parkinson's Disease.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e. a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose.; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the disclosure are contained in-between a card and clear PVC. The PVC can be transparent so the item (patch, pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (patch, pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed patches, pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e. Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

The transdermal formulation and/or topical formulation of the disclosure may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butyene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decymethylsulfoxide etc), dimethylisosorbide, mineral oils, vegetable oils, water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA), acids such as but not limited to acetic acid, lactic acid, levulinic acid, bases and others. More preferably in the range of 0.01%-95% w/w or w/v. In exemplary embodiments, formulations of the disclosure may comprise solvent(s) at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solvent(s) at a concentration of about 30 to 99%, of about 35% to 95%, about 40% to about 90% w/w. In exemplary formulations of the disclosure, the solvent(s) will represent approximately 1 wt % to 75 wt %, preferably 2 wt % to 30 wt %, more preferably 5 wt. % to 20 wt. % of the formulation.

### Example 2

The transdermal formulation and/or topical formulation of the disclosure may comprise gelling agents and/or thickening and/or suspending agents known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc, clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4501, 4202 etc.,), acrylic pressure sensitive adhesives such as but not limited to (duro-tak 87-2156, duro-tak 387-2287, etc.), polyisobutylene such as but not limited to (polyisobutylene low molecular weight, plyisobutylene medium molecular weight, polyisobutylene 35000 mw, etc), acrylic copolymers, rubber based adhesives, hot melt adhesives, styrene-butadiene copolymers, bentonite, all water and/or organic solvent swellable polymers, etc. More preferably in the range of 0.1% 70% w/w or w/v. In exemplary embodiments, formulations of the disclosure may comprise gelling agents and/or thickening and/or suspending agents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise gelling agents and/or thickening and/or suspending agents at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the gelling agents and/or thickening and/or suspending agents will represent approximately 1 wt % to 75 wt %, preferably 2 wt % to 30 wt %, more preferably 5 wt. % to 20 wt. % of the formulation.

### Example 3

The transdermal formulation and/or topical formulation of the disclosure may comprise penetration or permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc), 1,3-butanediol, azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, decyl oleate, glycerol monooleate, glycerol monolaurate, methyl laurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W Smith, Howard I. Maibach, 2005. November, CRC press). More preferably in the range of 0.01%-95% w/w or w/v. In exemplary embodiments, formulations of the disclosure may comprise permeation enhancer(s) at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise penetration or permeation enhancer(s) at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the permeation enhancer(s) will represent approximately 1 wt % to 75 wt %, preferably 2 wt % to 30 wt %, more preferably 5 wt. % to 20 wt. % of the formulation.

### Example 4

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). More preferably in the range of 0.01%-95% w/w or w/v. In exemplary embodiments, formulations of the disclosure may comprise plasticizer(s) at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizer(s) at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the plasticizer(s) will represent approximately 1 wt % to 75 wt %, preferably 2 wt % to 30 wt %, more preferably 5 wt. % to 20 wt. % of the formulation.

### Example 5

The transdermal formulation and/or topical formulation of the disclosure may comprise emollients, humectants, skin irritation reducing agents and the similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and the similar compounds or chemicals at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and the similar compounds or chemicals at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the emollients, humectants, skin irritation reducing agents and the similar compounds or chemicals will represent approximately 1 wt % to 75 wt %, preferably 2 wt % to 30 wt %, more preferably 5 wt. % to 20 wt. % of the formulation.

### Example 6

The transdermal formulation and/or topical formulation of the disclosure may comprise solubilizers, surfactants, emulsifying agents, dispersing agents and similar compounds or chemicals known to those skilled in the art either alone or in combination thereof without any limitation to following like polysorbate (e.g., TWEEN®) such as but not limited to (polysorbate 20, polysorbate 40, polysorbate 60, polysorbate 80, etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, ethyl oleate, polyglyceryl-3-dioleate, diethylene glycol monoethyl ether, propylene glycol monolaurate type I, polyglyceryl-3-dioleate, caprylocaproyl polyoxyl—8 glycerides etc, cyclodextrins, LABRASOL® (a caprylocaproyl macrogolglyceride, Caprylocaproyl macrogol-8 glycerides EP, Caprylocaproyl polyoxyl-8 glycerides NF), and others. More preferably in the range of 0.01% 95% w/w or w/v. In exemplary embodiments, formulations of the disclosure may comprise solubilizers, surfactants, emulsifying agents, dispersing agents and similar compounds or chemicals at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds or chemicals at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the solubilizers, surfactants, emulsifying agents, dispersing agents and similar compounds or chemicals will represent approximately 1 wt % to 75 wt %, preferably 2 wt % to 30 wt %, more preferably 5 wt. % to 20 wt. % of the formulation.

### Example 7

Different techniques and ingredients can be used to increase the stability and/or solubility of highly purified CBD in formulation such as without any limitation to coating, encapsulation, microencapsulation, nanoencapsulation, lyophilization, chelating agents, complexing agents, etc.

### Example 8

The transdermal formulation and/or topical formulation of the disclosure may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise auxiliary pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise auxiliary pH buffering agents and pH stabilizers and similar compounds at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the auxiliary pH buffering agents and pH stabilizers and similar compounds will represent approximately 1 wt % to 75 wt %, preferably 2 wt % to 30 wt %, more preferably 5 wt. % to 20 wt. % of the formulation. In certain embodiments, the pH of the formulation is maintained at about 4.0, about 4.5, about 5.0, about 5.5, about 6.0, about 6.5, about 7.0, about 7.5, or about 8.0. In certain embodiments, the pH of the formulation is maintained at a range of about 4.0 to about 8.0, about 4.5 to about 7.5, or about 5.0 to about 7.0.

### Example 9

The transdermal formulation and/or topical formulation of the disclosure may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation.

### Example 10

The transdermal formulation and/or topical formulation of the disclosure may be formulated in ointment and/or cream base known to those skilled in the art.

### Example 11

Materials to make the transdermal delivery system of the disclosure in patch form known to those skilled in the art, for example, such as but not limited to reservoir patch, matrix patch, drug in adhesives, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid-isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The transdermal formulation and/or topical formulation and/or transdermal delivery system of the disclosure may deliver at least therapeutic effective dose of highly purified CBD. Therapeutic effective highly purified CBD, alone or in combinations thereof in human plasma required for treating and/or preventing Parkinson's disease. Therapeutic effective highly purified CBD dose refers to the therapeutic concentration of in human plasma required for treating and/or preventing Parkinson's disease. Furthermore, the precise therapeutic effective dose of highly purified CBD in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

In yet another embodiment, the transdermal formulation and/or topical formulation and/or transdermal delivery system of the disclosure may deliver at least therapeutic effective dose of highly purified CBD. Therapeutic effective highly purified CBD refers to the therapeutic concentration of highly purified CBD thereof in human plasma required for the treatment and/or prevention and/or control of Parkinson's Disease in a patient.

The transdermal formulation or transdermal patch of highly purified CBD preferably but not limited to can be applied to the skin surface in any of the following dosage regimens such as once in a day, once in two days, once in three days, once in four days, once in five days, once in six days, once in a week, once in a 8 to about 13 days, once in two weeks, once in 15 days.

### Example 12

### Example Formulations of Drug in Adhesive Matrix Patch

| Component | % W/W |
|---|---|
| Active component (THC and/or CBD) | 0.5%-30% |
| Solvent | 2%-30% |
| Permeation enhancer | 2%-30% |
| Pressure sensitive adhesive polymer | 20%-80% |
| Polymer | 1%-10% |

### Example Formulations of Drug in Adhesive Matrix Patch

| Component | % W/W |
|---|---|
| Active component (THC and/or CBD) | 1%-30% |
| Solvent | 2%-30% |
| Permeation enhancer | 2%-30% |
| Pressure sensitive adhesive polymer | 20%-80% |

### Example 13

Synthetic delta-9-thc (THC) and cannabidiol (CBD) formulations for transdermal delivery ((Formulation Nos. 001, 002, 003, 004, and 005) were prepared by mixing ingredients as shown in Table 1:

TABLE 1
Transdermal Synthetic Cannabidiol formulations

| | 001 | 002 | 003 | 004 | 005 |
|---|---|---|---|---|---|
| Ingredients | (% W/W) | (% W/W) | (% W/W) | (% W/W) | (% W/W) |
| CBD | 8.0 | 8.0 | 7.7 | 6.9 | 6.4 |
| THC | 8.0 | 8.0 | 7.7 | 6.9 | 6.4 |
| Ethanol | 42.7 | 37.3 | 37.0 | 33.0 | 25.4 |
| Propylene Glycol | 40.0 | 40.0 | 37.0 | 33.0 | 30.5 |
| Isopropyl | | | 4.7 | — | — |
| Palmitate | | | | | |
| DMSO | | | | 13.8 | 12.7 |
| Oleic Acid | | 5.3 | 4.7 | 5.5 | 5.1 |
| NMP | | | | — | 12.7 |

Abbreviations: THCH = delta-9-THC; CBD = Cannabidiol; NMP: N-methyl Pyrrolidone.

All of the components from Table 1, with the exception of the CBD and THC, were mixed together with stirring for 18 hours. Next, the CBD and THC were added into the excipient mixture to prepare the final transdermal formulations.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD and THC formulations prepared as described above. The receptor compartment was filled with receptor medium, held at constant temperature, and constantly stirred to collect the CBD and THC as it diffuses through the skin and into receptor compartment. It is important to confirm that the receptor fluid is always in contact with the skin. The receptor compartment was emptied at 24 hr intervals for assay of CBD and THC and replaced with fresh receptor solution. In order to maintain the sink condition in receptor compartment, it is important to keep the CBD and THC concentration in receptor compartment less than 10% of its solubility. The exerimental conditions are provided in Table 2:

TABLE 2
Experimental Condition for In-vitro Permeability testing

| | De-ionized water + 0.5% Brij-O(20) + |
|---|---|
| Receiving Media | 0.01% Sodium Azide |
| Receiving Media Volume (mL) | 13 |
| Sample Volume (mL) | 13 |
| Sampling Interval (hr) | 24, 48, 72 |
| Franz-cell diffusion area (sqcm) | 1.76 |
| Membrane Type | Human |
| | Cadaver Skin |

Flux of CBD and THC through the human cadaver skin was measured for a minimum period of 72 Hrs (3 days) and results of the flux measurement are provided in Table 3 and 4.

TABLE 3
CBD Flux Results

| | 009 | 010 | 011 | 012 | 013 |
|---|---|---|---|---|---|
| Average Flux (0-24 hr) | 0.53 | 0.86 | 1.10 | 1.39 | 1.01 |
| (ug/sqcm/hr) | (23.6%) | (27.0%) | (20%) | (19.9%) | (5.1%) |
| Average Flux (24-48 hr) | 0.92 | 1.16 | 1.21 | 1.44 | 1.21 |
| (ug/sqcm/hr) | (9.6%) | (14.4%) | (18.3%) | (1.65%) | (27.3%) |
| Average Flux (48-72 hr) | 0.52 | 0.86 | 0.71 | 1.27 | 1.01 |
| (ug/sqcm/hr) | (2.44%) | (12.9%) | (13%) | (16.3%) | (27.5%) |
| Average Flux (0-72 hr) | 0.66 | 0.96 | 1.01 | 1.37 | 1.08 |
| (ug/sqcm/hr) | | | | | |

TABLE 4
THC Flux Results

| | 009 | 010 | 011 | 012 | 013 |
|---|---|---|---|---|---|
| Average Flux (0-24 hr) | 0.0 | 0.28 | 0.62 | 0.73 | 0.60 |
| (ug/sqcm/hr) | | (89%) | (20.4%) | (24.5%) | (5.1%) |
| Average Flux (24-48 hr) | 0.39 | 0.65 | 0.78 | 0.85 | 0.80 |
| (ug/sqcm/hr) | (87%) | (20.8%) | (19.4%) | (8.7%) | (32.3%) |
| Average Flux (48-72 hr) | 0.0 | 0.47 | 0.43 | 0.73 | 0.58 |
| (ug/sqcm/hr) | | (20.3%) | (32%) | (20.2%) | (40%) |
| Average Flux (0-72 hr) | 0.13 | 0.47 | 0.61 | 0.77 | 0.66 |
| (ug/sqcm/hr) | | | | | |

### Example 14

Synthetic cannabidiol (CBD) formulations for transdermal delivery ((Formulation Nos. 047-055) were prepared by mixing ingredients as shown in Table 5:

TABLE 5
Transdermal Synthetic Cannabidiol formulations

| Excipients | CBD 047 | CBD 048 | CBD 049 | CBD 050 | CBD 051 | CBD 052 | CBD 053 | CBD 054 | CBD 055 |
|---|---|---|---|---|---|---|---|---|---|
| CBD | 4.84% | 4.98% | 4.73% | 4.99% | 4.87% | 4.89% | 5.04% | 4.83% | 5.00% |
| DURO-TAK | 95.16% | — | — | — | — | — | — | — | — |
| 2516 | | | | | | | | | |
| DURO-TAK | — | 95.02% | — | — | — | — | — | — | — |
| 9301 | | | | | | | | | |
| DURO-TAK | — | — | 95.27% | — | — | — | — | — | — |
| 2287 | | | | | | | | | |
| DURO-TAK | — | — | — | 95.01% | — | — | — | — | — |
| 2054 | | | | | | | | | |
| DURO-TAK | — | — | — | — | 95.13% | — | — | — | — |
| 2852 | | | | | | | | | |
| DURO-TAK | — | — | — | — | — | 95.11% | — | — | — |
| 2074 | | | | | | | | | |
| DURO-TAK | — | — | — | — | — | — | 94.96% | — | — |
| 2194 | | | | | | | | | |
| BIO-PSA 4501 | — | — | — | — | — | — | — | 95.17% | — |
| BIO-PSA 4201 | — | — | — | — | — | — | — | — | 95.00% |

The above ingredients (Table 5) are blended by stirring for 18 hours and then, using a commercial benchtop spreader, the matrix is evenly spread onto an 8×14 inch sheet of release liner (such as 3M 9744) to a thickness of 0.5 mm. The sheet is then place in an oven at 86 F for 120 min to evaporate off the ethyl acetate adhesive solvent. An opaque backing membrane (such as 3M 9730 NR film) with low permeability to oxygen to inhibit photo and oxidative degradation, is then carefully applied by hand to avoid formation of bubbles and voids. A circular die (1.5 inches diameter) is used to cut patches (1.76 sqcm) for subsequent studies. After drying, the drug adhesive matrix has a surface density of 2-30 mg/sqcm, containing CBD in 5% w/w.

The prepared formulations where then subjected to a release study as follows: After weighing the patches (n=3), the release liner was removed, and the patches were placed in 20 ml scintillation vials with 15 ml of receiving media. The receiving media was PBS solution of pH 7.4 with 0.5% Brij(O)20. Vials were placed on the roller overnight at 20 RPM. Samples were withdrawn every 24 hours, up to 72 hours, and media was fully replaced each time. Samples were then run in the HPLC in order to determine the % release of CBD from the different formulations.

The prepared formulations also analyze for the uniformity of drug content. The patches (n=3) were weight out for each formulation, the release liner was removed, and the patches (including the release liner) were placed in 20 ml scintillation vials with 15 ml of solution IPA:Ethanol (190 proof) (50:50). The vials were then placed on the roller at 20 RPM and left overnight. Samples were withdrawn from each vial and analyzed on the HPLC in order to determine the drug content of each formulation.

TABLE 6
Experimental Condition for In-vitro Permeability testing

| | PBS (pH 7.4) + 0.5% Brij-O(20) + |
|---|---|
| Receiving Media | 0.01% Sodium Azide |
| Receiving Media Volume (mL) | 15 |
| Sample Volume (mL) | 15 |
| Sampling Interval (hr) | 24, 48 |
| Diffusion area (sqcm) | 1.76 |

% Release of CBD through the matrix system was measured for a minimum period of 48 Hrs (2 days) and results of the % release are provided in Table 7.

TABLE 7
% CBD Release Results

| | CBD 047 | CBD 048 | CBD 049 | CBD 050 | CBD 051 | CBD 052 | CBD 053 | CBD 054 | CBD 055 |
|---|---|---|---|---|---|---|---|---|---|
| Av. % | 26 (3) | 9 (7) | 20 (7) | 40 (2) | 23 (8) | 35 (12) | 37 (6) | 93 (2) | Did not |
| Release at 24 | | | | | | | | | solubilize |
| hours | | | | | | | | | the CBD |
| Av. % Release | 18 (7) | 6 (12) | 13 (13) | 22 (1) | 13 (9) | 12 (1) | 22 (5) | 2 (2) | 98% |
| at 48 hours | | | | | | | | | (0.05) |
| % | 44 | 15 | 33 | 62 | 36 | 47 | 59 | 95 | |
| Release | | | | | | | | | |
| 0-48 hours | | | | | | | | | |
| (cumulative) | | | | | | | | | |
| % CBD from | 104 (2) | 100 (1) | 100 (5) | 102 (1) | 103 (6) | 96 (15) | 104 (4) | 107 | |
| extraction | | | | | | | | | |
| (STD) | | | | | | | | | |

Drug content study showed that the recover of CBD in extraction is between 96-107% for all the manufactured formulations. Furthermore, the release study showed that the silicone adhesive 4501 showed more than 90% release within first 24 hrs. Based on the release profile following are the best adhesive for CBD formulation: BIOPSA-4501>2054=2194>2074>2516>2852=2287>9301.

Release studies indicate that the functional group and crosslinker affect the CBD release from acrylic adhesive. According to current study, acrylic adhesive containing —COOH functional group with crosslinker showed the maximum release of CBD from all the acrylic adhesive patches.

### Example 15

Additional synthetic Cannabidiol (CBD) formulations for transdermal delivery (Formulation Nos. 057 through 064) were prepared by mixing ingredients as shown in Table 8:

TABLE 8
Transdermal Synthetic Cannabidiol formulation no. 057 to 064

| Excipients | CBD 057 | CBD 058 | CBD 059 | CBD 060 | CBD 061 | CBD 062 | CBD 063 | CBD 064 |
|---|---|---|---|---|---|---|---|---|
| CBD | 5.0% | 5.0% | 5.0% | 4.9% | 4.8% | 4.8% | 4.9% | 4.8% |
| BIO-PSA 4501 | 95.0% | 84.6% | 84.5% | 85.0% | 83.3% | 89.7% | 86.9% | 89.6% |
| IPM | — | 10.4% | — | — | — | — | — | — |
| IPP | — | — | 10.5% | — | — | — | — | — |
| Oleic Acid | — | — | — | 10.1% | — | — | — | — |
| Transcutol P | — | — | — | — | 11.9% | — | — | — |
| Brij O20 | — | — | — | — | — | 5.5% | — | — |
| Poloxamer 124 | — | — | — | — | — | — | 8.2% | — |
| PGML | — | — | — | — | — | — | — | 5.6% |

Synthetic Cannabidiol formulations for transdermal delivery (057-064) were prepared by the same procedure described in Example 14.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD formulations prepared as described above. The receptor compartment was filled with receptor medium, held at constant temperature, and constantly stirred to collect the CBD as it diffuses through the skin and into receptor compartment. It is important to confirm that the receptor fluid is always in contact with the skin. The receptor compartment was emptied at 24 hr intervals for assay of CBD and replaced with fresh receptor solution. In order to maintain the sink condition in receptor compartment, it is important to keep the CBD concentration in receptor compartment less than 10% of its solubility. The experimental conditions are provided in Table 9:

TABLE 9
Experimental Condition for In-vitro Permeability testing

| | De-ionized water + 0.5% Brij-O(20) + |
|---|---|
| Receiving Media | 0.01% Sodium Azide |
| Receiving Media Volume (mL) | 13 |
| Sample Volume (mL) | 13 |
| Sampling Interval (hr) | 24, 48, 72, 96 |
| Franz-cell diffusion area (sqcm) | 1.76 |
| Membrane Type | Human Cadaver Skin |

Flux of CBD through the human cadaver skin was measured for a minimum period of 96 Hrs (4 days) and results of the flux measurement are provided in Table 10.

Upon completion of the flux study, the used patches were carefully removed and extract the CBD from the use patches using IPA:Ethanol (50:50). The human cadaver skin was also soaked in IPA:Ethanol (50:50), in order to extract the CBD from it. The samples were analyzed using HPLC. The data in table 6 showed the amount of CBD present in the skin and the left-over patches.

TABLE 10
CBD Flux Results

| | CBD 057 | CBD 058 | CBD 059 | CBD 060 | CBD 061 | CBD 062 | CBD 063 | CBD 064 |
|---|---|---|---|---|---|---|---|---|
| Av. cumulative | 52 | 31 | 16 | 120 | 86 | BLLQ | BLLQ | 53 |
| amount permeated | | | | | | | | |
| at 96 hrs (μg) | | | | | | | | |
| Av. flux 24- | 0.41 | 0.37 | 0.37 | 0.69 | 0.51 | BLLQ | BLLQ | 0.42 |
| 96 hrs (μg/hr/sqcm) | | | | | | | | |
| Peak flux | 0.44 | 0.38 | 0.38 | 0.82 | 0.60 | BLLQ | BLLQ | 0.44 |
| (μg/hr/sqcm) | | | | | | | | |
| Time to peak flux | 72 | 72 | 72 | 72 | 72 | 0 | 0 | 72 |
| (hrs) | | | | | | | | |
| Av. CBD Amount in | 1.57 | Did not Determined | | 1.14 | 2.26 | 1.81 | 1.88 | 1.56 |
| patch (mg) | | | | | | | | |
| Av. CBD Amount in | 0.01 | | | 0.59 | 0.13 | 0.54 | 0.24 | 0.10 |
| skin (mg) | | | | | | | | |

### Example 16

Additional synthetic Cannabidiol (CBD) formulations for transdermal delivery (Formulation Nos. 102 through 106) were prepared by mixing ingredients as shown in Table 11:

TABLE 11
Transdermal Synthetic Cannabidiol formulation no. 102 to 106

| Excipients | CBD-102 | CBD-103 | CBD-104 | CBD-105 | CBD-106 |
|---|---|---|---|---|---|
| CBD | 3.69% | 4.01% | 3.90% | 3.97% | 3.94% |
| Oleic acid | 3.69% | — | 4.30% | — | 9.06% |
| Propylene Glycol | 12.19% | 13.22% | 12.50% | 13.49% | — |
| Isopropyl Palmitate | — | 4.41% | 4.69% | — | 6.31% |
| BIO-PSA 4501 | 80.42% | 78.37% | 74.62% | 82.54% | 80.69% |

Synthetic Cannabidiol formulations for transdermal delivery (102-106) were prepared by the same procedure described in Example 14.

The in-vitro flux study was performed by the same procedure described in Example 15. Flux of CBD through the human cadaver skin was measured for a minimum period of 144 Hrs (6 days) and results of the flux measurement are provided in Table 8.

TABLE 12
CBD Flux Results

| Excipients | CBD-102 | CBD-103 | CBD-104 | CBD-105 | CBD-106 |
|---|---|---|---|---|---|
| Avg Flux 0- | 0.23 (0.37) | 0.51 (87.37) | 0.12 (173.21) | 0.13 (173.21) | 0.00 |
| 24 hr, μg/sqcm/hr (% RSD) | | | | | |
| Avg Flux 24- | 0.44 (8.00) | 0.30 (2.80) | 0.32 (14.52) | 0.31 (12.19) | 0.30 (22.80) |
| 90 hr, μg/sqcm/hr (% RSD) | | | | | |
| Avg Flux 90- | 0.53 (10.96) | 0.40 (13.95) | 0.41 (13.50) | 0.35 (13.20) | 0.36 (16.54) |
| 120 hr, μg/sqcm/hr (% RSD) | | | | | |
| Avg Flux 120- | 0.51 (9.02) | 0.42 (11.41) | 0.44 (8.42) | 0.27 (15.04) | 0.27 (87.40) |
| 144 hr, μg/sqcm/hr (% RSD) | | | | | |
| Avg Flux 24- | 0.43 (7.09) | 0.41 (28.88) | 0.32 (12.14) | 0.26 (13.47) | 0.23 (31.69) |
| 144 hr, μg/sqcm/hr (% RSD) | | | | | |

### Example 17

Additional synthetic Cannabidiol (CBD) formulations for transdermal delivery (Formulation No. 060) were prepared by mixing ingredients as shown in Table 13:

TABLE 13
Transdermal Synthetic Cannabidiol formulation no. 60

| Excipients | CBD-060 |
|---|---|
| CBD | 4.84% |
| Oleic acid | 10.01% |
| BIO-PSA 4501 | 85.15% |

Synthetic Cannabidiol formulations for transdermal delivery (060) were prepared by the same procedure described in Example 14.

The in-vitro flux study was performed by the same procedure described in Example 2. The effect of receiving media (RM) was evaluated using several different RM: 1) 0.5% w/w Brij 020 in water, 2) 1% w/w Brij O20 in water, 3) 0.5% w/w Brij O20 in water:Ethanol (50:50), 4) 0.5% w/w Brij O20 in Water:Ethanol (90:10), 5) 0.5% w/w Brij O20 in Water:Ethanol (80:20), 6) 0.5% w/w Brij O20 in Water:Ethanol (70:30) and, 7) 0.5% w/w Brij O20 in Water:PEG400 (60:40). Flux of CBD through the human cadaver skin was measured for a minimum period of 168 Hrs (7 days) and results of the flux measurement are provided in Table 14, Table 15 and Table 16.

TABLE 14
CBD Flux Results

| | | DI water + | DI water with 0.5% Brij |
|---|---|---|---|
| | DI water + | 1.0% Brij | O20 (50%) + ethanol |
| Receiving Media | 0.5% Brij O20 | O20 | (50%) |
| Avg Flux 0- | BLLQ | BLLQ | 6.81 (12.65) |
| 24 hr, μg/sqcm/hr (% RSD) | | | |
| Avg Flux 24- | BLLQ | BLLQ | 5.01 (10.46) |
| 48 hr, μg/sqcm/hr (% RSD) | | | |
| Avg Flux 48- | BLLQ | BLLQ | 3.73 (21.04) |
| 72 hr, μg/sqcm/hr (% RSD) | | | |
| Avg Flux 72- | BLLQ | BLLQ | 1.24 (21.31) |
| 96 hr, μg/sqcm/hr (% RSD) | | | |
| Avg Flux 96- | BLLQ | BLLQ | 4.14 (22.31) |
| 120 hr, μg/sqcm/hr (% RSD) | | | |
| Avg Flux 120- | BLLQ | BLLQ | 3.08 (31.94) |
| 144 hr, μg/sqcm/hr (% RSD) | | | |
| Avg Flux 144- | BLLQ | BLLQ | 0.55 (81.13) |
| 168 hr, μg/sqcm/hr (% RSD) | | | |

TABLE 15
CBD Flux Results

| | DI water with 0.5% | DI water with 0.5% | DI water with 0.5% |
|---|---|---|---|
| | Brij O20 (90%) + | Brij O20 (80%) + | Brij O20 (50%) + |
| Receiving Media | ethanol (10%) | ethanol (20%) | ethanol (50%) |
| Avg Flux 0- | BLLQ | 0.15 (154.97) | 5.58 (24.33) |
| 24 hr, μg/sqcm/hr (% RSD) | | | |
| Avg Flux 24- | 0.24 (111.0) | 0.13 (154.92) | 4.20 (11.02) |
| 48 hr, μg/sqcm/hr (% RSD) | | | |
| Avg Flux 48- | 0.15 (155.34) | 0.08 (244.95) | 5.16 (25.90) |
| 72 hr, μg/sqcm/hr (% RSD) | | | |
| Avg Flux 72- | 0.27 (77.80) | 0.08 (244.95) | 4.01 (18.19) |
| 96 hr, μg/sqcm/hr (% RSD) | | | |
| Avg Flux 96- | 0.49 (10.47) | 0.15 (155.68) | 3.37 (13.85) |
| 120 hr, μg/sqcm/hr (% RSD) | | | |
| Avg Flux 120- | 0.42 (49.57) | 0.16 (155.47) | 2.54 (29.27) |
| 144 hr, μg/sqcm/hr (% RSD) | | | |
| Avg Flux 144- | BLLQ | 0.18 (138.85) | 0.44 (99.0) |
| 168 hr, μg/sqcm/hr (% RSD) | | | |

TABLE 16
CBD Flux Results

| | DI water with 0.5% | DI water with 0.5% | |
|---|---|---|---|
| | Brij O20 (70%) + | Brij O20 (50%) + | DI water (60%) + |
| Receiving Media | ethanol (30%) | ethanol (50%) | PEG-400 (40%) |
| Avg Flux 0- | 0.36 (79.23) | 4.49 (37.81) | BLLQ |
| 24 hr, μg/sqcm/hr (% RSD) | | | |
| Avg Flux 24- | 1.23 (31.26) | 11.69 (8.97) | 0.49 (18.12) |
| 48 hr, μg/sqcm/hr (% RSD) | | | |
| Avg Flux 48- | 1.68 (27.58) | 13.07 (84.62) | 0.62 (14.70) |
| 72 hr, μg/sqcm/hr (% RSD) | | | |
| Avg Flux 72- | 1.32 (22.94) | 5.80 (33.13) | 0.62 (13.38) |
| 96 hr, μg/sqcm/hr (% RSD) | | | |
| Avg Flux 96- | 0.98 (20.19) | 3.19 (42.69) | 0.66 (14.62) |
| 120 hr, μg/sqcm/hr (% RSD) | | | |

### Example 18

Additional synthetic Cannabidiol (CBD) formulations for transdermal delivery (Formulation No. 102 and 104) were prepared by mixing ingredients as shown in Table 17:

TABLE 17
Transdermal Synthetic Cannabidiol formulation no. 102 and 104

| Excipients | CBD-102 | CBD-104 |
|---|---|---|
| CBD | 4.34 | 4.21 |
| PG | 13.45 | 13.04 |
| Oleic Acid | 4.34 | 4.63 |
| IPP | — | 4.21 |
| BIO-PSA 4501 | 77.86 | 73.91 |

Synthetic Cannabidiol formulations for transdermal delivery (102 and 104) were prepared by the same procedure described in Example 14.

The in-vitro flux study was performed by the same procedure described in Example 2. The effect of receiving media (RM) was evaluated using two different RM: 1) 0.5% w/w Brij O20 in water, and 2) 0.50 w/w Brij O20 in water:Ethanol (50:50. Flux of CBD through the human cadaver skin was measured for a minimum period of 120-144 Hrs (5-6 days) and results of the flux measurement are provided in Table 18.

TABLE 18
CBD Flux Results

| | CBD-102 | | CBD-104 | |
|---|---|---|---|---|
| | | DI water with | | DI water with |
| | | 0.5% Brij O20 | | 0.5% Brij O20 |
| Formulation | DI water + 0.5% | (50%) + ethanol | DI water + 0.5% | (50%) + ethanol |
| Receiving Media | Brij O20 | (50%) | Brij O20 | (50%) |
| Avg Flux 0-24 hr, | BLLQ | 4.10 (29.62) | BLLQ | 8.54 (9.22) |
| μg/sqcm/hr (% RSD) | | | | |
| Avg Flux 24-48 hr | BLLQ | 9.94 (1.99) | BLLQ | 9.67 (12.07) |
| μg/sqcm/hr (% RSD) | | | | |
| Avg Flux 48-72 hr, | 0.52 (10.97) | 13.87 (1.61) | BLLQ | 5.62 (24.78) |
| μg/sqcm/hr (% RSD) | | | | |
| Avg Flux 72-96 hr, | 0.42 (10.54) | 2.80 (4.14) | BLLQ | 3.66 (40.98) |
| μg/sqcm/hr (% RSD) | | | | |
| Avg Flux 96-120 hr, | 0.40 (7.27) | 16.27 (13.12) | BLLQ | 4.66 (45.26) |
| μg/sqcm/hr (% RSD) | | | | |
| Avg Flux 120-144 hr, | — | — | BLLQ | 2.20 (61.51) |
| μg/sqcm/hr (% RSD) | | | | |

While the invention has been described in detail and with reference to specific examples thereof, it will be apparent to one skilled in the art that various changes and modifications can be made therein without departing from the spirit and scope thereof.

What is claimed is:

1. A method of treating, and/or slowing the onset or progression of Parkinson's disease (PD) and/or a related symptom in a patient comprising:
selecting a patient in need of treating, and/or slowing the onset or progression of PD and/or a related symptom;
topically applying a pharmaceutical composition in the form of a transdermal matrix patch comprising:
synthetic delta-9 tetrahydrocannabinol (THC), wherein the concentration of the synthetic delta-9 THC in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w, and/or
synthetic cannabidiol (CBD), wherein the concentration of the synthetic CBD in the pharmaceutical composition is selected from the group consisting of about 0.5%, about 1%, about 2%, about 3%, about 4%, about 5%, about 6%, about 7%, about 8%, about 9%, about 10%, about 11%, about 12%, about 13%, about 14%, and about 15% w/w,
wherein the pharmaceutical composition further comprises:
about 5% to about 15% w/w of a solvent comprising propylene glycol;
about 6% to about 15% w/w of a penetration enhancer comprising oleic acid;
about 50% to about 80% w/w of a silicone pressure sensitive adhesive;
about 5% to about 15% w/w of at least one suspending agent comprising silicon dioxide;
about 0.1%, 0.2%, 0.3%, 0.4%, 0.5% or 0.6% of an antioxidant comprising BHT,
wherein the transdermal matrix patch provides an average flux of the synthetic delta-9 THC of about 0.47 to about 0.85 μg/cm²/hr over at least 7 days,
thereby treating, and/or slowing the onset or progression of PD and/or a related symptom in the patient.

2. The method of claim 1 wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL, about 2 μg/mL, and about 5 μg/mL.

3. The method of claim 1 wherein the pharmaceutical composition is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

4. The method of claim 1 wherein the pharmaceutical composition is co-administered with at least one additional an active agent selected from the group consisting of: a dopamine precursor, a dopamine receptor agonist, and combinations thereof.

5. The method of claim 1 wherein the pharmaceutical composition is co-administered with at least one additional an active agent selected from the group consisting of: levodopa, bromocriptine, pergolide, pramipexole, cabergoline, ropinorole, apomorphine, and any combination thereof.

6. The method of claim 1 wherein the symptom of PD to be treated, and/or slowed is selected from the group consisting of:
(a) at least one non-motor aspect of experiences of daily living as defined by Part I of the Unified Parkinson's Disease Rating Scale selected from the group consisting of cognitive impairment, hallucinations and psychosis, depressed mood, anxious mood, levodopa induced nausea, apathy, features of dopamine dysregulation syndrome, sleep problems, daytime sleepiness, pain, urinary problems, constipation problems, lightheadedness on standing, and fatigue;
(b) at least one motor aspect of experiences of daily living as defined by Part II of the Unified Parkinson's Disease Rating Scale selected from the group consisting of speech, saliva and drooling, chewing and swallowing, eating tasks, dressing, hygiene, handwriting, turning in bed, tremors, getting out of a bed, a car, or a deep chair, walking and balance, and freezing;
(c) at least one motor symptom identified in Part III of the Unified Parkinson's Disease Rating Scale selected from the group consisting of speech, facial expression, rigidity, finger tapping, hand movements, pronation-supination movements of hands, toe tapping, leg agility, arising from chair, gait, freezing of gait, postural stability, posture, body bradykinesia, postural tremor of the hands, kinetic tremor of the hands, rest tremor amplitude, and constancy of rest tremor;
(d) at least one motor complication identified in Part IV of the Unified Parkinson's Disease Rating Scale selected from the group consisting of time spent with dyskinesias, functional impact of dyskinesias, time spent in the off state, functional impact of fluctuations, complexity of motor fluctuations, and painful off-state dystonia;
(e) constipation;
(f) depression;
(g) cognitive impairment;
(h) short or long term memory impairment;
(i) concentration impairment;
(j) coordination impairment;
(k) mobility impairment;
(l) speech impairment;
(m) mental confusion;
(n) sleep problem, sleep disorder, or sleep disturbance;
(o) circadian rhythm dysfunction;
(p) hallucinations;
(q) fatigue;
(r) REM disturbed sleep;
(s) REM behavior disorder;
(t) erectile dysfunction;
(u) postural hypotension;
(v) correction of blood pressure or orthostatic hypotension;
(w) nocturnal hypertension;
(x) regulation of temperature;
(y) improvement in breathing or apnea;
(z) correction of cardiac conduction defect;
(aa) amelioration of pain;
(bb) urinary incontinence, or restoration of bladder sensation and urination;
(cc) mood swings;
(dd) apathy;
(ee) control of nocturia;
(ff) neurodegeneration;
(gg) anxiety, including anxiety related to public speaking;
(hh) improving speaking ability, including public speaking ability; and/or
(ii) Parkinson's Disease Dementia.

7. The method of claim 1 wherein the PD symptom to be treated, and/or slowed is a sleep problem, sleep disorder, sleep disturbance, circadian rhythm dysfunction, REM disturbed sleep, or REM behavior disorder, and wherein:
(a) the sleep disorder or sleep disturbance comprises a delay in sleep onset, sleep fragmentation, REM-behavior disorder, sleep-disordered breathing including snoring and apnea, day-time sleepiness, micro-sleep episodes, narcolepsy, hallucinations, or any combination thereof, and/or
(b) the REM-behavior disorder comprises vivid dreams, nightmares, and acting out the dreams by speaking or screaming, or fidgeting or thrashing of arms or legs during sleep; and/or
(c) treating the sleep problem, sleep disorder, or sleep disturbance delays the onset and/or progression of the Parkinson's disease; and/or
(d) the method results in a positive change in the sleeping pattern of the subject over a defined period of time; and/or
(e) the method results in a positive change in the sleeping pattern of the subject over a defined period of time, wherein the positive change is defined as:
(i) an increase in the total amount of sleep obtained of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the number of awakenings during the night selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(f) as a result of the method the subject obtains the total number of hours of sleep recommended by a medical authority for the age group of the subject; and/or
(g) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

8. The method of claim 1 wherein the PD symptom to be treated, and/or slowed is hallucination and wherein:
(a) the hallucination comprises a visual, auditory, tactile, gustatory or olfactory hallucination; and/or
(b) treating the hallucination delays the onset and/or progression of the Parkinson's disease; and/or
(c) the method results in a decreased number of hallucinations of the subject over a defined period of time; and/or
(d) the method results in a decreased number of hallucinations of the subject over a defined period of time and the decrease in number is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(e) the method results in the subject being hallucination-free; and/or
(f) the method results in a decreased severity of hallucinations of the subject over a defined period of time, as measured by one or more medically recognized technique; and/or
(g) the method results in a decreased severity of hallucinations of the subject over a defined period of time and the decrease in severity is selected from the group consisting of by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%, as measured by one or more medically recognized technique; and/or
(h) the medically recognized technique selected from the group consisting of Chicago Hallucination Assessment Tool (CHAT), The Psychotic Symptom Rating Scales (PSYRATS), Auditory Hallucinations Rating Scale (AHRS), Hamilton Program for Schizophrenia Voices Questionnaire (HPSVQ), Characteristics of Auditory Hallucinations Questionnaire (CAHQ), Mental Health Research Institute Unusual Perception Schedule (MUPS), positive and negative syndrome scale (PANSS), scale for the assessment of positive symptoms (SAPS), Launay-Slade hallucinations scale (LSHS), the Cardiff anomalous perceptions scale (CAPS), and structured interview for assessing perceptual anomalies (SIAPA); and/or
(i) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

9. The method of claim 1 wherein the PD symptom to be treated, and/or slowed is depression and wherein:
(a) treating the depression delays the onset and/or progression of the Parkinson's disease; and/or
(b) the method results in improvement in a subject's depression over a defined period of time, as measured by one or more clinically-recognized depression rating scale; and/or
(c) the method results in improvement in a subject's depression over a defined period of time, as measured by one or more clinically-recognized depression rating scale and the improvement is in one or more depression characteristics selected from the group consisting of mood, behavior, bodily functions such as eating, sleeping, energy, and sexual activity, and/or episodes of sadness or apathy; and/or
(d) the method results in improvement in a subject's depression, as measured by one or more clinically-recognized depression rating scale, and the improvement a subject experiences following treatment is about 5, about 10, about 15, about 20, about 25, about 30, about 35, about 40, about 45, about 50, about 55, about 60, about 65, about 70, about 75, about 80, about 85, about 90, about 95 or about 100%; and/or
(e) wherein each defined period of time is independently selected from the group consisting of about 1 day to about 10 days, about 10 days to about 30 days, about 30 days to about 3 months, about 3 months to about 6 months, about 6 months to about 12 months, and about greater than 12 months.

10. The method of claim 1 wherein the PD symptom to be treated, and/or slowed is cognitive impairment, and wherein:
(a) treating the cognitive impairment delays the onset and/or progression of the Parkinson's disease; and/or
(b) progression or onset of the cognitive impairment is slowed, halted, or reversed over a defined period of time following administration of the pharmaceutical composition, as measured by a medically-recognized technique; and/or
(c) the cognitive impairment is positively impacted by the administration of the pharmaceutical composition, as measured by a medically-recognized technique; and/or
(d) the cognitive impairment is positively impacted by the administration of the pharmaceutical composition, as measured by a medically-recognized technique and the positive impact on and/or progression of cognitive decline is measured quantitatively or qualitatively by one or more techniques selected from the group consisting of Mini-Mental State Exam (MMSE), Mini-cog test, and a computerized tested selected from Cantab Mobile, Cognigram, Cognivue, Cognision, or Automated Neuropsychological Assessment Metrics; and/or
(e) the progression or onset of cognitive impairment is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(f) each defined period of time is independently selected from the group consisting of about 1 day to about 10 days, about 10 days to about 30 days, about 30 days to about 3 months, about 3 months to about 6 months, about 6 months to about 12 months, and about greater than 12 months.

11. The method of claim 1 wherein the PD symptom to be treated, and/or slowed is constipation, and wherein:
(a) treating the constipation delays the onset and/or progression of the Parkinson's disease; and/or
(b) the administration of the pharmaceutical composition causes the subject to have a bowel movement; and/or
(c) the method results in an increase in the frequency of bowel movement in the subject over a defined period of time; and/or
(d) the method results in an increase in the frequency of bowel movement in the subject and the increase in the frequency of bowel movement is defined as:
(i) an increase in the number of bowel movements per week of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%; and/or
(ii) a percent decrease in the amount of time between each successive bowel movement selected from the group consisting of about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%; and/or
(e) as a result of the method the subject has the frequency of bowel movement recommended by a medical authority for the age group of the subject.

12. The method of claim 1 wherein the PD symptom to be treated, and/or slowed is neurodegeneration correlated with PD, and wherein:
(a) treating the neurodegeneration delays the onset and/or progression of the Parkinson's disease; and/or
(b) the method results in treating, and/or delaying the progression and/or onset of neurodegeneration in the subject; and/or
(c) progression or onset of the neurodegeneration is slowed, halted, or reversed over a defined period of time following administration of the pharmaceutical composition, as measured by a medically-recognized technique; and/or
(d) the neurodegeneration is positively impacted by the administration of the pharmaceutical composition, as measured by a medically-recognized technique; and/or
(e) the positive impact and/or progression of neurodegeneration is measured quantitatively or qualitatively by one or more techniques selected from the group consisting of electroencephalogram (EEG), neuroimaging, functional MRI, structural MRI, diffusion tensor imaging (DTI), [18F]fluorodeoxyglucose (FDG) PET, agents that label amyloid, [18F]F-dopa PET, radiotracer imaging, volumetric analysis of regional tissue loss, specific imaging markers of abnormal protein deposition, multimodal imaging, and biomarker analysis; and/or
(f) the progression or onset of neurodegeneration is slowed, halted, or reversed by about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%, as measured by a medically-recognized technique; and/or
(g) each defined period of time is independently selected from the group consisting of about 1 day to about 10 days, about 10 days to about 30 days, about 30 days to about 3 months, about 3 months to about 6 months, about 6 months to about 12 months, and about greater than 12 months.
